(19) United States
(12) Patent Application Publication
(10) Pub. No.: US 2025/0043036 A1
(43) Pub. Date: Feb. 6, 2025

(54) Chain Transfer Agents and Methods

(71) Applicant: Chevron Phillips Chemical Company LP, The Woodlands, TX (US)

(72) Inventors: Jason L. Kreider, Bartlesville, OK (US); Dale M. Solaas, Borger, TX (US); Kenneth M. Lassen, Bartlesville, OK (US); Rodney S. Childress, Bartlesville, OK (US)

(73) Assignee: Chevron Phillips Chemical Company LP, The Woodlands, TX (US)

(21) Appl. No.: 18/782,431

(22) Filed: Jul. 24, 2024

Related U.S. Application Data
(60) Provisional application No. 63/515,685, filed on Jul. 26, 2023.

(51) Int. Cl. C08F 2/38; C07C 321/04; C08F 2/22; C08F 112/08

(52) U.S. Cl. CPC C08F 2/38; C07C 321/04; C08F 2/22; C08F 112/08

(57) ABSTRACT

Chain transfer agent compositions that can include mixed mercaptans. Emulsion polymerization reaction mixtures that can include a chain transfer agent composition, one or more monomers, and one or more polymerization initiators. Methods of polymerization, which can include providing an emulsion polymerization reaction mixture, and isolating a polymer product from the emulsion polymerization reaction mixture.

### CROSS-REFERENCE TO RELATED APPLICATIONS

This application claims priority to U.S. Provisional Patent Application No. 63/515,685, Jul. 26, 2023, which is incorporated by reference herein.

### TECHNICAL FIELD OF THE DISCLOSURE

This disclosure relates to compositions, such as chain transfer agent compositions, that include mixed mercaptans, and the use of chain transfer agent compositions in chemical reactions, such as emulsion polymerization reactions.

### BACKGROUND

A number of mercaptans, such as tertiary dodecyl mercaptan (TDM), n-dodecyl mercaptan (NDDM), and other C₈ to C₁₄ mercaptans, are used as chain transfer agents. The production rates, however, of some mercaptans, such as TDM, can be inhibited for one or more reasons, such as the desire or need to reach high SH content.

There remains a need for chain transfer agents or compositions that exhibit improved performance, allow for a higher SH content to be reached, and/or include mixed mercaptans that can increase the capacity of other mercaptans, such as TDM, by increasing the SH content while enhancing or not undesirably impacting performance.

### SUMMARY

This summary is provided to introduce various concepts in a simplified form that are further described below in the detailed description. This summary is not intended to identify required or essential features of the claimed subject matter nor is the summary intended to limit the scope of the claimed subject matter.

Provided herein are chain transfer agent compositions, which can include mixed mercaptans. In one aspect, a chain transfer agent composition is provided that can include a first component and a second component. The first component can include tert-dodecyl mercaptan, tert-nonyl mercaptan, n-dodecyl mercaptan, or any combination thereof, such as a mixture of tert-dodecyl mercaptan and tert-nonyl mercaptan; and the second component can include one or more branched alkyl mercaptans, one or more branched alkyl sulfides, or a combination thereof. In some embodiments, at least one of the one or more branched alkyl mercaptans of the second component is not tert-dodecyl mercaptan and is not tert-nonyl mercaptan.

Also provided herein are emulsion polymerization reaction mixtures. In some embodiments, the emulsion polymerization reaction mixtures include at least one of the chain transfer agent compositions provided herein, one or more monomers, one or more polymerization initiators, optionally one or more surfactants, and water. A chain transfer agent composition can be present in an emulsion polymerization reaction mixture at any amount, such as about 0.01 wt % to about 5 wt %, based on a total weight of the emulsion polymerization mixture.

In yet another aspect, methods of polymerization are provided. In some embodiments, the methods include providing an emulsion polymerization reaction mixture as described herein; and optionally isolating a polymer product from the emulsion polymerization reaction mixture.

Additional aspects will be set forth in part in the description which follows, and in part will be obvious from the description, or may be learned by practice of the aspects described herein. The advantages described herein may be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

FIG. 1 depicts a first Mayo plot of embodiments of a polymerization of styrene in the presence of tert-dodecyl mercaptan.

FIG. 2 depicts a second Mayo plot of embodiments of a polymerization of styrene in the presence of tert-dodecyl mercaptan.

FIG. 3 depicts a first Mayo plot of embodiments of a polymerization of styrene in the presence of tert-nonyl mercaptan.

FIG. 4 depicts a second Mayo plot of embodiments of a polymerization of styrene in the presence of tert-nonyl mercaptan.

FIG. 5 depicts a first Mayo plot of embodiments of a polymerization of styrene in the presence of a mixture of tert-nonyl and tert-dodecyl mercaptans.

FIG. 6 depicts a second Mayo plot of embodiments of a polymerization of styrene in the presence of a mixture of tert-nonyl and tert-dodecyl mercaptans.

FIG. 7 depicts a first Mayo plot of embodiments of a polymerization of styrene in the presence of n-dodecyl mercaptan.

FIG. 8 depicts a second Mayo plot of embodiments of a polymerization of styrene in the presence of n-dodecyl mercaptan.

FIG. 9 depicts a first Mayo plot of embodiments of a polymerization of styrene in the presence of MMIO 1.

FIG. 10 depicts a second Mayo plot of embodiments of a polymerization of styrene in the presence of MMIO 1.

FIG. 11 depicts a first Mayo plot of embodiments of a polymerization of styrene in the presence of MMIO 2.

FIG. 12 depicts a second Mayo plot of embodiments of a polymerization of styrene in the presence of MMIO 2.

FIG. 13 depicts a first Mayo plot of embodiments of a polymerization of styrene in the presence of MMIO 3.

FIG. 14 depicts a second Mayo plot of embodiments of a polymerization of styrene in the presence of MMIO 3.

FIG. 15 depicts a first Mayo plot of embodiments of a polymerization of styrene in the presence of MMIO 4.

FIG. 16 depicts a second Mayo plot of embodiments of a polymerization of styrene in the presence of MMIO 4.

FIG. 17 depicts a plot of weight average molecular weight of polystyrene versus the percentage of styrene conversion for embodiments of emulsion polymerizations conducted in the absence of a chain transfer agent.

FIG. 18 depicts a plot of weight average molecular weight of polystyrene versus the percentage of styrene conversion for embodiments of emulsion polymerizations conducted in the presence of 0.25 wt % MMIO 2.

FIG. 19 depicts a plot of weight average molecular weight of polystyrene versus the percentage of styrene conversion for embodiments of emulsion polymerizations conducted in the presence of 0.5 wt % MMIO 2.

FIG. 20 depicts a plot of weight average molecular weight of polystyrene versus the percentage of styrene conversion for embodiments of emulsion polymerizations conducted in the presence of 0.25 wt % tert-dodecyl mercaptan.

FIG. 21 depicts a plot of weight average molecular weight of polystyrene versus the percentage of styrene conversion for embodiments of emulsion polymerizations conducted in the presence of 0.5 wt % tert-dodecyl mercaptan.

FIG. 22 depicts a plot of weight average molecular weight of polystyrene versus the percentage of styrene conversion for embodiments of emulsion polymerizations conducted in the presence of 0.25 wt % MMIO 4.

FIG. 23 depicts a plot of weight average molecular weight of polystyrene versus the percentage of styrene conversion for embodiments of emulsion polymerizations conducted in the presence of 0.5 wt % MMIO 4.

FIG. 24 depicts a plot of (M_{w})MMIO 2/M_{w} tert-dodecyl mercaptan versus the percentage of styrene conversion at 0.5 wt % of the chain transfer agents.

FIG. 25 depicts a plot of (M_{w})MMIO 4/M_{w} tert-dodecyl mercaptan versus the percentage of styrene conversion at 0.5 wt % of the chain transfer agents.

FIG. 26 depicts a plot of (M_{w})MMIO 4/M_{w} tert-dodecyl mercaptan versus the percentage of styrene conversion at 0.25 wt % of the chain transfer agents.

FIG. 27 depicts a plot of number average molecular weight at full conversion versus chain transfer level (%).

FIG. 28 depicts a plot of weight average molecular weight at full conversion versus chain transfer level (%).

FIG. 29 depicts a plot of weight average molecular weight versus the percentage of styrene conversion in the presence of a chain transfer agent including 100% tert-dodecyl mercaptan.

FIG. 30 depicts a plot of weight average molecular weight versus the percentage of styrene conversion in the presence of a chain transfer agent including 1:3 MMIO 2: tert-dodecyl mercaptan.

FIG. 31 depicts a plot of weight average molecular weight versus the percentage of styrene conversion in the presence of a chain transfer agent including 1:1 MMIO 2: tert-dodecyl mercaptan.

FIG. 32 depicts a plot of weight average molecular weight versus the percentage of styrene conversion in the presence of a chain transfer agent including 3:1 MMIO 2: tert-dodecyl mercaptan.

FIG. 33 depicts a plot of weight average molecular weight versus the percentage of styrene conversion in the presence of MMIO 2.

FIG. 34 depicts a plot of weight average molecular weight versus the percentage of MMIO 2 in a mixture of MMIO 2 and tert-dodecyl mercaptan at the end of each reaction.

### DETAILED DESCRIPTION

Provided herein are chain transfer agent compositions, emulsion polymerization reaction mixtures, and methods of emulsion polymerization. The emulsion polymerization reaction mixtures can include any of the chain transfer agent compositions provided herein.

### Chain Transfer Agent Compositions

Compositions, such as chain transfer agent compositions, are provided herein. The compositions can include a first component and a second component. The first component and the second component can be present at any weight ratio in a chain transfer agent composition. In some embodiments, the first component and the second component are present at a weight ratio (first component: second component) of about 10:90 to about 99:1, about 20:80 to about 99:1, about 30:70 to about 99:1, about 40:60 to about 99:1, about 50:50 to about 99:1, about 60:40 to about 99:1, about 70:30 to about 99:1, about 80:20 to about 99:1, or about 90:10 to about 99:1. In some embodiments, the second component and the first component are present at a weight ratio (second component: first component) of about 10:90 to about 99:1, about 20:80 to about 99:1, about 30:70 to about 99:1, about 40:60 to about 99:1, about 50:50 to about 99:1, about 60:40 to about 99:1, about 70:30 to about 99:1, about 80:20 to about 99:1, or about 90:10 to about 99:1.

The first component can include tert-dodecyl mercaptan, tert-nonyl mercaptan, n-dodecyl mercaptan, or a combination thereof, such as a mixture of tert-dodecyl mercaptan and tert-nonyl mercaptan.

The second component can include alkyl mercaptans, such as one or more branched alkyl mercaptans, one or more alkyl sulfides, such as one or more branched alkyl sulfides, or a combination thereof. In some embodiments, at least one of the one or more alkyl mercaptans of the second component is not tert-dodecyl mercaptan and/or is not tert-nonyl mercaptan.

The one or more branched alkyl mercaptans can include any of those known in the art. The term “branched”, as used herein to describe mercaptans, sulfides, or other compounds, is used in a manner that is consistent with its plain and ordinary meaning in the relevant art, and generally refers to any hydrocarbyl, e.g., alkyl, group that includes one or more tertiary carbon atoms at any position(s); therefore, for example, the phrase “a branched C₁₀ alkyl” includes and reads on all constitutional isomers that include 10 carbon atoms. In some embodiments, the one or more branched alkyl mercaptans includes one or more branched C₅ to C₂₀ mercaptans, one or more branched C₅ to C₁₅ mercaptans, one or more branched C₈ to C₁₂ mercaptans, one or more branched C₉ to C₁₁ mercaptans, or one or more branched C₁₀ mercaptans. In some embodiments, the second component includes (i) one or more branched C₁₀ mercaptans, and (ii) one or more C₁₂ mercaptans, one or more C₁₄ mercaptans, one or more C₁₆ mercaptans, one or more C₁₈ mercaptans, or a combination thereof. The one or more branched alkyl mercaptans can be present at any effective amount, such as an amount of at least about 60 wt %, at least about 70 wt %, at least about 80 wt %, at least about 85 wt %, or at least about 90 wt %, based on the weight of the second component.

In some embodiments, the one or more alkyl mercaptans include one or more mercaptans of the following formula—

R—SH;

wherein R is independently selected from a C₅ to C₂₀ alkyl, a C₅ to C₁₅ alkyl, a C₈ to C₁₂ alkyl, a C₉ to C₁₁ alkyl, or a C₁₀ alkyl, such as, respectively, a branched C₅ to C₂₀ alkyl, a branched C₅ to C₁₅ alkyl, a branched C₈ to C₁₂ alkyl, a branched C₉ to C₁₁ alkyl, or a branched C₁₀ alkyl. When one compound of the foregoing formula is present, each R can be identical, however, when two or more compounds of the foregoing formula are present, each R is “independently” selected from the recited options, meaning that an R group of a first compound and an R group of a second compound can be different; for example, the R groups of the first and second compounds can be a branched C₉ alkyl and a branched C₁₀ alkyl, respectively, or, as a further example, two different isomers of a branched C₁₀ alkyl (see, e.g., “Structure A” and “Structure B” below). The term “independently” applies in a similar manner to other substituents herein, such as R¹ and R².

In some embodiments, the one or more branched alkyl mercaptans include a compound of Structure A, a compound of Structure B, a compound of Structure C, a compound of Structure D, a compound of Structure E, a compound of Structure F, a compound of Structure G, a compound of Structure H, or a combination thereof:

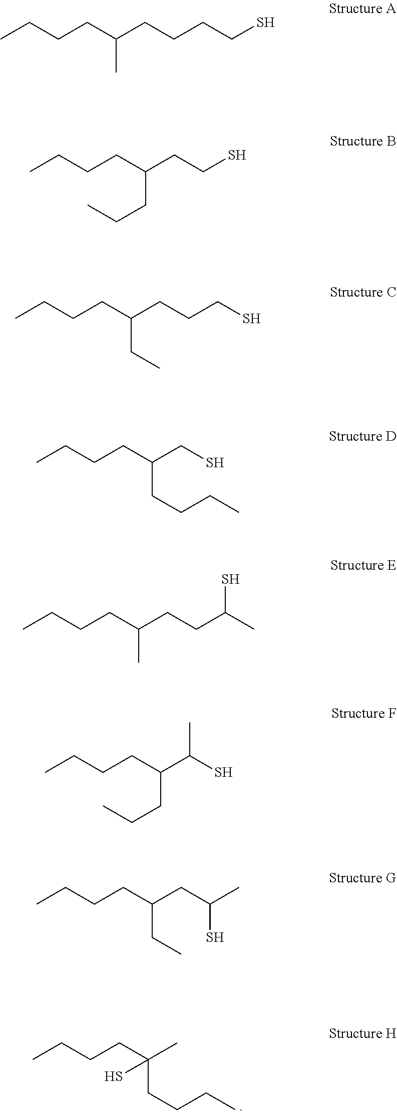

The one or more alkyl sulfides can include any of those known in the art. In some embodiments, the one or more alkyl sulfides includes one or more branched C₁₀ to C₄₀ sulfides, one or more branched C₁₀ to C₃₀ sulfides, or one or more branched C₂₀ sulfides. In some embodiments, the one or more alkyl sulfides includes one or more compounds of the following formula:

R¹—S—R²;

wherein (i) each R¹ and R² is independently selected from a C₅ to C₂₀ alkyl, a C₅ to C₁₅ alkyl, a C₈ to C₁₂ alkyl, a C₉ to C₁₁ alkyl, or a C₁₀ alkyl, such as, respectively, a branched C₅ to C₂₀ alkyl, a branched C₅ to C₁₅ alkyl, a branched C₈ to C₁₂ alkyl, a branched C₉ to C₁₁ alkyl, or a branched C₁₀ alkyl; or (ii) R¹ and R² are independently selected from the group consisting of the following:

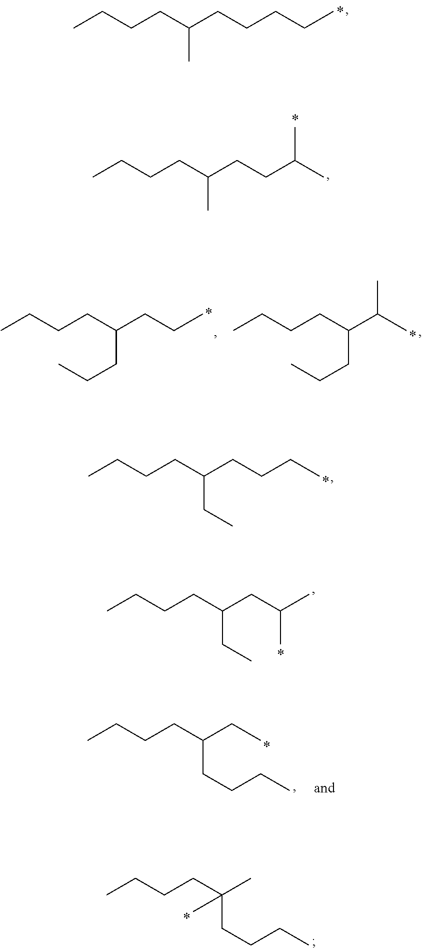

wherein “*” designates attachment points of the “S” atom. For example, when the following structure is selected for R¹:

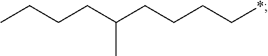

the remaining portion of the formula would have the following structure in view of the position of the asterisk (“*”):

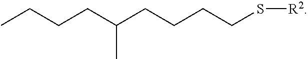

The one or more branched alkyl sulfides can be present at any effective amount, such as an amount of less than about 40 wt %, less than about 30 wt %, less than about 20 wt %, less than about 15 wt %, or less than about 10 wt %, based on the weight of the second component.

In some embodiments, the one or more branched alkyl mercaptans, the one or more branched alkyl sulfides, or a combination thereof are the reaction products of contacting hydrogen sulfide (H₂S), a feedstock including one or more branched C₅ to C₂₀ monoolefins, an initiating agent, and/or a catalyst. The one or more branched C₅ to C₂₀ monoolefins can include branched C₁₀ monoolefins, such as 5-methyl-1-nonene, 3-propyl-1-heptene, 4-ethyl-1-octene, 2-butyl-1-hexene, or combinations thereof. The crude composition can include branched C₁₀ mercaptans and branched C₂₀ sulfides.

In some embodiments, the second component includes MMIO 1, MMIO 2, MMIO 3, MMIO 4, or a combination thereof. In some embodiments, the first component includes tert-dodecyl mercaptan, and the second component includes MMIO 1, MMIO 2, MMIO 3, MMIO 4, or a combination thereof. In some embodiments, the first component includes tert-nonyl mercaptan, and the second component includes MMIO 1, MMIO 2, MMIO 3, MMIO 4, or a combination thereof. In some embodiments, the first component includes n-dodecyl mercaptan, and the second component includes MMIO 1, MMIO 2, MMIO 3, MMIO 4, or a combination thereof. In some embodiments, the first component includes a mixture of tert-dodecyl mercaptan and tert-nonyl mercaptan, and the second component includes MMIO 1, MMIO 2, MMIO 3, MMIO 4, or a combination thereof.

### Polymerization Reaction Mixtures

Also provided herein are polymerization reaction mixtures, such as emulsion polymerization reaction mixtures. The polymerization reaction mixtures can include any chain transfer agent composition provided herein; optionally one or more monomers; optionally one or more surfactants; optionally one or more polymerization initiators; and optionally water.

A chain transfer agent composition can be present in a polymerization reaction mixture at any effective amount, such as an amount of about 0.01 wt. % to about 5 wt. %, about 0.01 wt. % to about 4 wt. %, about 0.01 wt. % to about 3 wt. %, about 0.01 wt. % to about 2 wt. %, or about 0.01 wt. % to about 1 wt. %, based on a total weight of the emulsion polymerization mixture. A second component of a chain transfer agent composition can be present at any effective amount in a polymerization reaction mixture, such as an amount of at least 10 wt. %, at least 20 wt. %, or at least 30 wt. % based on a total weight of the chain transfer agent composition.

The one or more monomers of a polymerization reaction mixture can include any monomer(s) known in the art. The one or more monomers can be of the same type, to form a homopolymer, or different types, to form a copolymer. The one or more monomers can include a monomer that is capable of free radical polymerization.

### Methods of Polymerization

Also provided herein are methods of polymerization. The methods can include providing a polymerization mixture, such as an emulsion polymerization mixture; and isolating a polymer product from the polymerization mixture. The polymerization mixture can include a chain transfer agent composition provided herein.

The isolation of a polymer product from a polymerization mixture, such as an emulsion polymerization mixture, can be achieved using any known technique, including, but not limited to, filtration, extraction, etc.

The polymer product can include a polymer. The term “polymer”, as used herein, refers to and includes any molecule formed by covalently bonding two or more monomers together, such as an oligomer, homopolymer, copolymer, etc., of any configuration, e.g., linear, branched, crosslinked, star, comb, etc.

### EXAMPLES

The disclosure is further illustrated by the following examples, which are not to be construed in any way as imposing limitations to the scope of this disclosure. Various other aspects, embodiments, modifications, and equivalents thereof which, after reading the description herein, can suggest themselves to one of ordinary skill in the art without departing from the spirit of the present disclosure or the scope of the appended claims.

In the following examples, four samples of mixed mercaptans were used, which are arbitrarily labeled MMIO 1, MMIO 2, MMIO 3, and MMIO 4. These four samples were prepared according to the following procedures, which are described in U.S. Patent Nos. 9,512,071, 9,738,601, 9,938,237, 10,011,564, 10,040,758, 9,512,248, 9,631,039, 9,879,102, and 10,000,590, which are incorporated by reference herein.

As described in the following procedures, MMIO 1 is a UV kettle product that is not distilled and is stripped only of the lights fraction. MMIO 2 is the product of H₂S reacted with an olefin feedstock over an acid catalyst, such as FILTROL® 24X acid catalyst (BASF, USA). MMIO 3 is the product of H₂S reacted with an olefin feedstock in the presence of a hydrodesulfurization catalyst, such as a cobalt molybdenum on alumina catalyst. MMIO 4 is a distilled UV product.

Hydrogen sulfide (H₂S) and a feedstock comprising branched C₁₀ monoolefins were reacted in the presence of various initiating agents and/or catalysts: UV radiation, an acid catalyst, and a hydrodesulfurization (HDS) catalyst.

Various feedstocks (e.g., olefin feedstocks) were used for reacting with H₂S to produce mercaptans and/or sulfides. More specifically, C₁₀ monoolefin feedstocks obtained from 1-hexene production processes were used as feedstocks for reacting with H₂S to produce mercaptans.

Gas chromatography-mass spectrometry (GC-MS) and nuclear magnetic resonance (NMR) spectroscopy were used for analyzing the composition of olefin feedstocks obtained from 1-hexene production processes as well as the products of the reaction of the olefin feedstocks with H₂S.

The compositions comprising C₁₀ monoolefins were analyzed by gas chromatography-mass spectrometry (GC-MS) using a 15 m×0.25 mm×0.5 μm DB-5 column and/or a 40 m×0.1 mm×0.1 μm DB-1 column to determine component identities and standard gas chromatography (GC) using a 60 m×0.32 mm×1 μm DB-1 column to determine the quantity of the components present in the compositions. These compositions were measured in area %, which is substantially similar and analogous to wt. %. Table 1 provides representative information about the typical composition of such an olefin feedstock obtained from 1-hexene production processes and reacted with H₂S to produce mercaptans.

TABLE 1
Composition of Mixed Decene Olefin Feedstock

| Chemical | GC Area % | | | Normalized % |
|---|---|---|---|---|
| Cyclohexane | 2.148 | | | |
| Octane | 0.036 | C₈ olefins | 1.17 | 1.24 |
| 1-octene | 1.135 | | | |
| Octane | 0.146 | Octane | 0.15 | 0.16 |
| Ethylbenzene | 1.684 | | | |
| 3-propyl-1-heptene | 14.590 | C₁₀ olefins | 84.16 | 89.11 |
| Decene | 0.164 | | | |
| 4-ethyl-1-octene | 13.134 | | | |
| 5-methyl-1-nonene | 32.144 | | | |
| Decene | 0.647 | | | |
| 2-butyl-1-hexene | 9.960 | | | |
| Decene | 0.320 | | | |
| 4/5 decene | 9.116 | | | |
| 1-decene | 4.086 | | | |
| Decane | 0.360 | Decane | 0.36 | 0.38 |
| 2-ethyl-1-hexanol | 1.379 | | | |
| Dodecene isomers | 0.448 | C₁₂ olefins | 1.29 | 1.37 |
| 1-Dodecene | 0.842 | | | |
| Dodecane | 0.182 | Dodecane | 0.18 | 0.19 |
| Tetradecenes | 6.710 | C₁₄ olefins | 6.71 | 7.11 |
| Tetradecane | 0.198 | Tetradecane | 0.2 | 0.21 |
| Octadecene | 0.222 | C₁₈ olefins | 0.22 | 0.23 |
| 2-ethylhexyl-2- | 0.069 | | | |
| ethylhexanoate | | | | |
| Unknowns | 0.281 | | | |
| Total | 100.000 | Total Olefins | 94.44 | 99.06 |

Normalized to include only octane, decane, dodecane, tetradecane, and C₈, C₁₀, C₁₂, C₁₄, and C₁₈ olefins

As indicated in Table 1, the total olefin content of this particular olefin feedstock (excluding the compounds that are not products of the 1-hexene process) sample is 94.44 area %, and 84.16 area % C₁₀ olefin isomers. The C₁₀ olefins represent over 89 area % of the total olefin content when the sample was normalized to remove the compounds that were not products of the 1-hexene process. Cyclohexane, ethylbenzene, and 2-ethylhexanol can be present in the olefin feedstock as residual components of the 1-hexene oligomerization process. The names and structures of C₁₀ isomers that can be present in the olefin feedstock are shown in the second columns of Table 2A and Table 2B, respectively.

TABLE 2A
Mixed Decene Olefins and Mercaptan Reaction Products

| Decene | | UV Reaction | Acid Catalyst |
|---|---|---|---|
| Fraction | Olefin | Products | Reaction Products |
| 5-methyl-1- nonene 32.14% (38.19) | 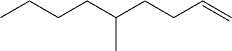 | 5-methyl-1- mercapto-nonane | 5-methyl-2-mercapto- nonane |
| 3-propyl-1- heptene 14.59% (17.33) | 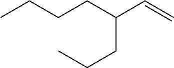 | 3-propyl-1- mercapto-heptane | 3-propyl-2-mercapto- heptane |
| 4-ethyl-1- octene 13.13% (15.60) | 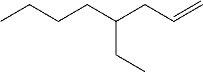 | 4-ethyl-1-mercapto- octane | 4-ethyl-2-mercapto- octane |
| 2-butyl-1- hexene 9.96% (11.83) | 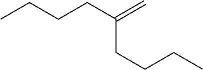 | 2-butyl-1-mercapto- hexane | 5-methyl-5-mercapto- nonane |
| 4/5-decene 9.12% (10.83) | 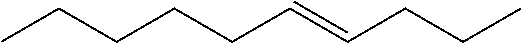 | 4-mercapto-decane 5-mercapto-decane | 4-mercapto-decane 4-mercapto-decane |
| | 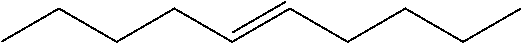 | | |
| 1-decene 4.09% | 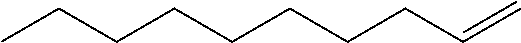 | 1-mercapto-decane | 2-mercapto-decane |
| (4.86) | | | |

TABLE 2B
Structures of Mercaptan Reaction Products

| Mercaptan | |
|---|---|
| Product | Structure |
| 5-methyl-1- mercapto-nonane | 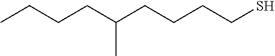 |
| 5-methyl-2- mercapto-nonane | 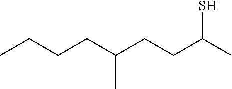 |
| 3-propyl-1- mercapto- heptane | 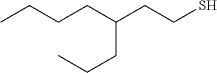 |
| 3-propyl-2- mercapto- heptane | 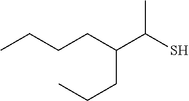 |
| 4-ethyl-1- mercapto-octane | 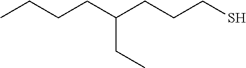 |
| 4-ethyl-2- mercapto-octane | 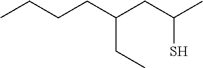 |
| 2-butyl-1- mercapto-hexane | 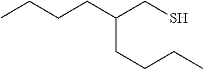 |
| 5-methyl-5- mercapto-nonane | 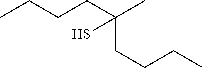 |
| 4-mercapto- decane | 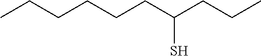 |
| 5-mercapto- decane | 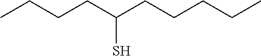 |
| 1-mercapto- decane | 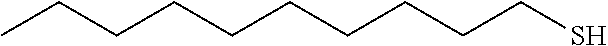 |
| 2-mercapto- decane | 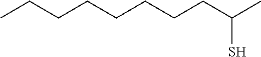 |

Referring to Table 2A and Table 2B, the first column of Table 2A provides the name of the isomer, the GC area % of that component in the feedstock from Table 1, and the normalized amount of the isomer typically found in just the C₁₀ fraction of the feedstock. Table 2B also displays the structure of the mercaptans that were produced from the C₁₀ olefin isomers. The second column of Table 2A shows the major C₁₀ olefin isomers in the feedstock; the third column of Table 2A lists the major mercaptan isomers produced by a UV-initiated reaction with H₂S; and the fourth column of Table 2A lists the major mercaptan isomers produced by acid catalysis. The structures of these compounds are depicted at Table 2B.

A sample of the olefin feedstock was fractionated (e.g., distilled) and only the C₁₀ fraction was isolated in high purity (e.g., a purified feedstock). This product was submitted for ¹Hand ¹³C NMR. The NMR analysis (in mol %) was consistent with the information provided by GC-MS. The NMR confirmed that about 11 mol % of the total was vinylidene (2 butyl-1-hexene isomer) and about 11 mol % of the total purified feedstock was internal olefins (linear decene isomers).

Reaction of H₂S with the olefin feedstock (e.g., a feedstock comprising branched C₁₀ monoolefins) by UV initiation (e.g., using UV radiation) yielded mostly primary mercaptans, since the terminal olefin and vinylidene isomers yield predominately the anti-Markovnikov product. The minor components were the secondary mercaptans from the terminal olefin and a tertiary mercaptan from the vinylidene isomer. UV-initiation of a terminal olefin produced primary mercaptans in 92-96 area % range and secondary mercaptans in 4-8 area % range. The linear internal olefin isomers present in the feedstock primarily produced secondary mercaptan isomers. Thus, for the composition of the feedstock disclosed herein, the distribution of mercaptans (i.e., the distribution within the C₁₀ fraction) in the resulting reaction product was predominately primary mercaptans comprising 80-90 area %. Secondary mercaptans were present at 10-20 area %, and tertiary mercaptans were present at about 0-3 area %. These ranges were calculated by NMR analysis of the reaction product.

Reaction of H₂S with the olefin feedstock over an acid catalyst (such as FILTROL® 24 or FILTROL® 24X acid catalyst (BASF, USA)), produced as the major product the Markovnikov product. The major mercaptan isomers contained secondary mercaptans with some tertiary mercaptan. The relative ratio of mercaptans was estimated at 85-90% secondary mercaptan and 10-15% tertiary mercaptan.

Reaction of H₂S with a feedstock comprising branched C₁₀ monoolefins in the presence of a hydrodesulfurization (HDS) catalyst (such as Haldor Topsoe TK-554 or TK-570) produced mercaptans similar in distribution to those produced by acid catalysis, which is the Markovnikov distribution. However, the HDS catalyst also produced a significant amount of the anti-Markovnikov product depending on the conditions used in the reaction step. Thus, under the conditions evaluated for this disclosure, the product produced by the HDS catalyst was a blend of the product produced via acid catalysis with some of the components produced by the UV-initiated reaction.

As will be appreciated by one of skill in the art, and with the help of this disclosure, the actual composition of the reaction product can ultimately depend on a number of factors: the exact composition of the feedstock, the ratio of H₂S to olefin that is used to produce the thiols, the catalytic method used to react the H₂S and olefin (UV vs. acid catalysis vs. HDS catalysis) to produce the product, etc. The final product (e.g., any cuts separated from the crude to form, for example, a commercial product) can also depend on the purification step to remove lights and whether a final product containing both mercaptan and sulfide fractions is desired or just one of the fractions, e.g., a mercaptan fraction or a sulfide fraction, is desired.

H₂S to Olefin Ratio: The H₂S to olefin molar ratio can be an important parameter in determining the amount of mercaptan and sulfide produced during the reaction step. This can be true regardless of the catalytic method employed. Without wishing to be limited by theory and in general, the higher the H₂S to olefin molar ratio, the greater the amount of mercaptans that can be produced compared to the amount of sulfides produced.

A general reaction scheme for addition of H₂S to an olefin is shown in the following scheme, regardless of catalytic method.

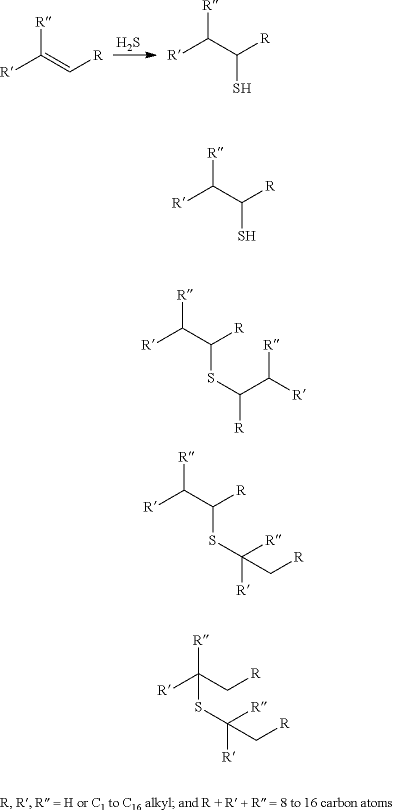

For a C₁₀ olefin fraction, R, R′and R″ can be H or C₁ to C₈ with the total of R+R′+R″=8. For 1-decene, R and R′=H and R″=8 and can be a linear or branched alkyl group. For the major isomers in a C₁₀ olefin fraction (e.g., a second feedstock as disclosed herein), 5-methyl-1-nonene: R and R′=H and R″=8, but the alkyl group contains branching at the third carbon atom of the C₈ fraction.

A sulfide fraction can be produced by further reaction of a mercaptan isomer with an olefin. The generic structures of such sulfides are shown herein, and this fraction can consist of a variety of isomers with several possible combinations of sulfide structures depending on whether the sulfide is primary to primary, primary to secondary, primary to tertiary, secondary to secondary, secondary to tertiary, or tertiary to tertiary. The structures can be complicated by the fact that on the two portions of the sulfide the R, R′ and R″ value can be the same or different, depending on which mercaptan isomer reacts with which olefin isomer. The total number of carbon atoms of the two portions of the sulfide can also have different values for R+R′+R″, although the most dominant combination will be where both sides each have a sum of 8 since the C₁₀ fraction predominates in the first feedstock and in the second feedstock.

Mercaptan Preparation Procedure: The tertiary dodecyl mercaptan (TDDM) used in these examples were commercial product samples obtained from the Chevron Phillips facility in Borger, Tex. The mixed C₁₀ mercaptan compositions were prepared using the following procedures.

Reaction Conditions: Three different reaction methods were used to perform the reaction of H₂S with a feedstock comprising branched C₁₀ monoolefins: UV initiation, acid catalysis, and HDS catalysis.

H₂S Removal: In laboratory experimentation, H₂S was removed using a rotary evaporator apparatus under conditions of reduced pressure. Under these conditions, H₂S was removed without removing significant quantities of light compounds.

Analytical Methods: The weight percentage of thiol (mercaptan) sulfur (wt. %

SH) was determined analytically by titration using iodine in water as the titrant and methylene chloride/isopropanol as the solvent system. Such titration can also be done by using a silver nitrate titration method. Total sulfur was measured by X-ray using a model SLFA-20 Horiba sulfur-in-oil analyzer. GC analysis was performed using an Agilent Technologies 7890A GC with a flame ionization detector. A 2 m×0.25 mm×1.0 m film DB-1 capillary column was used for the separation. Operating conditions were as follows: 70° C. initial temperature. 2 min hold time. 8° C./min ramp rate to 200° C. and then 15° C./min ramp rate to 300° C. and hold for 10 minutes. A 2 mL/min helium flow rate at constant flow conditions was used. The injector temperature was set at 275° C. and the detector temperature at 300° C. As described previously, these data from these compositions were reported in area %, which was substantially similar and analogous to wt. %. Olefin conversion was monitored using Raman spectroscopy, with a Kaiser Optical System RXN2 4-channel spectrometer. The peak centered at 1640 cm⁻¹ was the vinyl olefin, while the peak centered at about 1670 cm⁻¹ was the internal olefin.

UV Initiation: Reactions were performed using either a 1.5 L or a 5-liter UV reactor equipped with a 100 watt lamp and ballast. The two reactors were substantially the same configuration, and the only difference in operation was the amount of reactants added to the reactor. The reaction mixture was stirred at 500-1,000 RPM. The reaction temperature was controlled with a bath set at 25° C., but the heat of reaction reached about 40° C. The lamp operated at 1.1-1.5 amps and 28-103 volts over the course of the reaction, operating at lower amps and higher voltage as it warmed up. The reaction pressure was 220-280 psig (1,516 kPag-1,930 kPag) during the actual reaction time. The H₂S: olefin molar ratios were varied from 1.0 to 10.2; however, in theory, any H₂S to olefin ratio could be used. The reaction was completed in about 30 minutes based on the results of Raman Spectroscopy but was allowed to continue for 60 minutes to ensure completion. The main isomers from this reaction are listed in Table 3. Collected were gas chromatogram results of the crude reaction product resulting from the UV-initiated process after the removal of H₂S.

The relative amounts of C₁₀ mercaptan isomers, intermediate mercaptans and sulfide heavies depended on the ratio of HAS to olefin feedstock during the reaction step. Conventional wisdom would suggest that the C₁₀ mercaptan fraction would have too strong of an odor to be acceptable for certain applications, and that the sulfide fraction might have a better odor. Surprisingly and unexpectedly, after removing these samples from the reactor and venting off the residual H₂S using a rotary evaporator, the odor of this crude product was good. The limited odor of these compositions was an unexpected result that makes these compositions advantageous for use as chain transfer agents.

The composition of the UV-produced product can be described in broad terms as follows, taking into account that the crude reaction product can be subsequently separated into different fractions of different compositions and purity. In broad terms, the product consists of three general fractions as produced from the kettle product after removal of the unwanted lights fraction. Gas chromatogram results of the reaction product resulting from the UV-initiated process were collected following removal of the light fraction. The C₁₀ mercaptan fraction comprised from 50-100 wt. % of the crude kettle composition. The mercaptan functionality of the C₁₀ mercaptan fraction was 80-90% primary mercaptan, 5-18% secondary mercaptan and 0-3% tertiary mercaptan. This was the fraction that eluted in the 3.8-6.5 minute range under the GC conditions used. The intermediate fraction, which eluted in the 6.5-14 minute region, was predominately mercaptan isomers in the C₁₂ to C₁₈ range with a distribution of functionality that is similar to that for the C₁₀ isomer fraction. The intermediate fraction comprised from 0 to 12 area % of the kettle product. The heavy fraction (>14 minute retention time) consisted essentially of sulfides, primarily of formula C₁₀H₂₁—S—C₁₀H₂₁ isomers, as well as sulfides from C₁₂, C₁₄, C₁₆ or C₁₈ olefins and mercaptans or the asymmetric sulfides produced from the various combinations. These sulfide components comprised from 0-70 area % of the composition of the crude product.

Acid Catalysis: Acid catalyzed reactions produced a different distribution of isomer products than obtained by UV-initiation reaction of H₂S and the olefin feedstock comprising branched C₁₀ monoolefins.

The product produced via the acid catalyzed addition of H₂S to the feedstock comprising branched C₁₀ monoolefins was prepared in a continuous flow reactor over FILTROL® 24 acid catalyst. The reactor contained 43.22 g of catalyst and the WHSV (weight hourly space velocity) was maintained at 1.0 grams of olefin per gram of catalyst per hour. The H₂S to olefin molar ratio ranged from 10:1 to 1:1. The reaction temperature was between 120° C. to 220° C., and the reactor pressure was 450-460 psig (3,100 kPag-3,200kPag). Optimum results, based on conversion and maximum C10 mercaptan were achieved in the 180-200° C. range and at an HeS to olefin molar ratio of 5:1. A decrease in the HeS to olefin ratio resulted in a decrease in the C₁₀ mercaptan fraction and a corresponding increase in the sulfide fraction. Gas chromatogram analysis was conducted for the crude reaction product resulting from the acid-catalyzed process as compared to that produced by the UV-initiated process. Acid catalysis produced the Markovnikov product. The vinyl components of the feedstock comprising branched Cho monoolefins produced secondary mercaptans. The internal olefin components produced secondary mercaptans, while the vinylidene components produced tertiary mercaptans. Thus, the composition of the C₁₀ mercaptan fraction isomers was different when compared to the composition of the product obtained by UV-initiation. For example, the 5-methyl-1-nonene isomer produced 5-methyl-2-mercapto-nonane by acid catalysis; and 5-methyl-1-mercapto-nonane was the major product produced via UV-initiation, with a minor amount of the 2-mercapto isomer as a by-product. The 2-butyl-1-hexene isomer produced 5-methyl-5-mercapto-nonane via acid catalysis; while UV-initiation produced 2-butyl-1-mercapto-hexane.

As with the product produced via UV-initiation, the product obtained by acid catalysis consisted of three general fractions produced as kettle product after removal of the unwanted lights fraction. The C₁₀ mercaptan fraction included from 50-100 wt. % of the crude kettle composition. The mercaptan functionality of the C₁₀ fraction was 85-95% secondary mercaptan and the remainder tertiary mercaptan. These isomers eluted in the 3.1-6.5 minute range under the utilized GC conditions.

The intermediate fraction consisted of mercaptan peaks in the 6.5-14 minute range. However, the functionality of the mercaptans was secondary and tertiary C₁₂ to C₁₈ mercaptans. The intermediate fraction comprised 5-15% of the total kettle composition.

The sulfide fraction comprised 0-70% of the composition of the kettle product. The fraction consisted of sulfides primarily of formula C₁₀H₂₁—S—C₁₀H₂₁. However, the isomer identity was different than the product produced via UV-initiation. The acid catalyzed sulfide product was based on secondary and tertiary mercaptans rather than predominately primary mercaptans as in the UV-initiated product.

For both the UV-initiated and acid catalyzed product, purified samples of C₁₀ mercaptans were prepared. The purified C₁₀ mercaptan samples were prepared via distillation using a 52″ column packed with stainless steel packing. The first 7 fractions removed from the crude reaction product were considered to be the light fraction. This distillation step was considered to be complete when the kettle temperature increased from 100° C. to 121° C. and the head temperature increased from room temperature to 98.9° C. Cuts 8 to 13 were considered to be the intermediate fraction and included the purified C₁₀ mercaptans. These cuts were collected at a kettle temperature of 122° C. to 154° C. and a head temperature of 99° C.f to 105° C. Cuts 8 to 11 from the UV-initiated product and cuts 5to 11 from the acid catalyzed product were used for evaluation as chain transfer agents. Gas chromatograms representative of the typical purified C₁₀ mercaptan compositions (with lights, intermediates other than C₁₀ mercaptans, and heavies removed) from both the UV-initiated and acid catalyzed reactions were collected.

HDS Catalysis: Reactions utilizing HDS catalysis produced mercaptans that were primarily similar in distribution to those produced by acid catalysis, which is the Markovnikov distribution. However, there was a tendency to also produce some of the anti-Markovnikov distribution depending on the specific conditions utilized in the reaction step. Thus, the product produced by the HDS catalyst appeared to be a blend of product produced primarily via acid catalysis with some of the components of the UV-initiated reaction.

The HDS reaction conditions were as follows: WHSV was varied from 0.75 to 2 grams of olefin per gram of catalyst per hour; the molar ratio of H₂S per olefin was varied from 2:1 to 10:1; the average reaction temperature was 180° C. to 220° C. The catalyst used was cobalt molybdenum on alumina, examples being to Haldor Topsoe TK-554, TK-570, or similar. Olefin conversion, as determined by Raman Spectroscopy, was in the 88-97 mol % range.

Under similar conditions of WHSV, ratio and temperature, the HDS catalyzed reaction produced more C₁₀ mercaptan fraction and less sulfide fraction than the acid catalyzed reaction. Comparison of the GC analysis of the crude reaction product produced from the HDS catalyzed reaction of H₂S and branched C₁₀ monoolefins showed that the HDS-catalyzed reaction produced a crude reaction product that was a blend of the product compositions produced by the UV-initiated and acid-catalyzed reactions. Gas chromatogram analysis was performed on the crude reaction product (with only H₂S removed) resulting from the HDS catalyzed reaction.

In the following examples, a series of eight mercaptans were used in the bulk polymerization of styrene monomer initiated by azobisisobutyronitrile (AIBN) at 70° C. in solution polymerization. The purpose was to determine the chain transfer constants, CS, for all of the thiols as they interacted with the polystyrene free radical in the control of the polymer molecular weight.

Samples of the reacting fluid were withdrawn at times of reaction so that the degree of conversion was consistent at ˜5% for all experiments in the following examples. In addition to determining the monomer conversion in the samples, full molecular weight distributions and molecular weight averages were also measured via size exclusion chromatography (GPC). These data were used to create plots of molecular weight (actually the inverse of the degree of polymerization) versus the molar ratio of the mercaptan and monomer concentrations; the slopes of the linear fits to these data were then taken as the chain transfer constants.

The resulting values for the well-known mercaptans (e.g., tertiary dodecyl mercaptan (TDM), C_{S}=3.9+/−0.5) used in the study were close to those reported in the literature, which indicated that the experimental and analysis methods of the following examples is appropriate. The inventive mercaptans yielded CS values ranging from about 4 to about 9, with MMIO 2 being the same as TDM, and MMIO 4 being about twice that value. The CS values relate directly to the basic chemical activity of the mercaptans.

The emulsion polymerization reactions were designed to test the efficacy of all the mercaptans when used in water based latex production. The additional mechanism of molecular transport of the mercaptans from an emulsified droplet phase through the water, and then into the reacting latex particles allows for consideration of the water solubilities of the mercaptans. Styrene emulsion polymerizations (EP) were conducted using different mass ratios of mercaptan to monomer, but with consistent emulsion polymerization recipes and process conditions, all at 70° C. The EP studies were directed to MMIO 2 (which has a CS approximately equal to that of TDM), MMIO 4 (which has a CS approximately twice that of TDM) and TDM. Single mercaptan experiments were conducted at two concentration levels. Both MMIO mercaptans produced marked molecular weight reductions compared to zero mercaptan level, but both showed significant monomer conversion dependencies of the MW, unlike TDM. Not wishing to be bound by any particular theory, it is believed that the likely higher water solubility of the MMIO 2 and, particularly, MMIO 4 as compared to TDM, was a contributing cause of the monomer conversion dependent results. The MW results at the end of the latex reactions for all three mercaptans (used at the same level) were nearly the same. MMIO 2 and TDM mixtures were also studied in styrene EP at mass ratios of 3:1, 1:1, and 1:3, MMIO 2: TDM, but all with a combined mercaptan level of 0.5% on monomer. The results showed that the conversion dependency of the MMIO 2 was mitigated, and that the final MW was lower than using pure TDM at the 0.5% level. The results at the 1:1 ratio of MMIO 2: TDM were particularly encouraging.

### Example 1—Solution Polymerizations

The bulk polymerization systems of this example were designed so as to make the transfer constant analyses free of complicating features. Therefore, pure styrene monomer (including the inhibitor), azobisisobutyronitrile (AIBN) initiator, and the selected mercaptans were the systems studied. The reaction temperature was 70° C. Table 4 lists the sample designations, their molecular weights, and the material purities.

TABLE 4
Chain Transfer Agents

| | Molecular | Purity (% by |
|---|---|---|
| Material | Weight | mass) |
| Tert-dodecyl mercaptan (TDM) | 204 | 99+ |
| Tert-nonyl mercaptan (TNM) | 162 | 99+ |
| Mixture of tert-nonyl and tert-dodecyl | 177 | 99+ |
| mercaptans) (TMM) | | |
| n-dodecyl mercaptan (NDDM) | 202 | 99+ |
| MMIO 1 | 188 | 99+ |
| MMIO 2 | ~190 (mixture) | 99+ |
| MMIO 3 | ~190 (mixture) | 99+ |
| MMIO 4 | ~185 (mixture) | ~85 |
| Styrene monomer | 104 | 99+ |
| AIBN | 164 | 99.2 |

The reactions were carried out in narrow, sealed, glass capillary tubes and the polymer molecular weights were determined by size exclusion chromatography (GPC) using polystyrene standards. Twenty-seven reactions were run with the parameters provided in Table 5.

TABLE 5
Mercaptan Concentrations

| | Mercaptan | |
|---|---|---|
| Run | Concentration | Mercaptan |
| 1 | 0.0005 | TDM |
| 2 | 0.0005 | MMIO 1 |
| 3 | 0.0005 | MMIO 1 |
| 4 | 0.0005 | TNM |
| 5 | 0.005 | TDM |
| 6 | 0.005 | NDDM |
| 7 | 0.005 | MMIO 1 |
| 8 | 0.0005 | MMIO 2 |
| 9 | 0.0005 | MMIO 3 |
| 10 | 0.0025 | TDM |
| 11 | 0.005 | NDDM |
| 12 | 0.0005 | NDDM |
| 13 | 0.0025 | MMIO 3 |
| 14 | 0.005 | TMM |
| 15 | 0.0025 | TMM |
| 16 | 0.005 | TNM |
| 17 | 0.0005 | TMM |
| 18 | 0.005 | MMIO 1 |
| 19 | 0.0005 | NDDM |
| 20 | 0.0005 | TNM |
| 21 | 0.005 | MMIO 3 |
| 22 | 0.005 | TNM |
| 23 | 0.0025 | MMIO 2 |
| 24 | 0.005 | MMIO 2 |
| 25 | 0.0025 | TNM |
| 26 | 0.0025 | NDDM |
| 27 | 0.0025 | MMIO 1 |

The following methodology was used to determine the transfer constants provided in the examples. Given that the polymer chain length created during a free radical polymerization of vinyl monomers involves radical propagation and termination reactions, and radical transfer reactions, it was necessary to relate that chain length to the chemical and thermal parameters that control it. This was done by defining the number average chain length, X_{N}, as the rate of chain propagation divided by the total rate of stopping events (termination+transfer). X_{N} (also called the degree of polymerization, DP) is simply the number average molecular weight, M_{N} (measured experimentally), divided by the monomer molecular weight. In equation form (and inverted) this is expressed as follows:

The first term accounts for chain terminations (via recombination only) via the rate coefficients for termination, Kₜ, initiator dissociation, K_{d}, and propagation, Kₚ, all temperature dependent. The composition dependence of the termination reactions is related to the concentrations of initiator, [I], and monomer, [M]. Collectively this grouping is 1/DP₀, or the degree of polymerization without considering any chain transfer reactions. The second term in the equation accounts for chain stopping events due to an added chain transfer agent, such as a mercaptan. The ratio of rate coefficients (K_{tr,CTA}/Kₚ) is the “transfer constant”, often designated as C_{S}. This chain stopping term also includes the composition parameters [CTA] (e.g., mercaptan concentration) and [M]. The above equation is usually re-written as follows, and is referred to as the Mayo equation:

$$1 / DP = 1 / {DP}_{0} + C s [ C TA ] / [ M ]$$

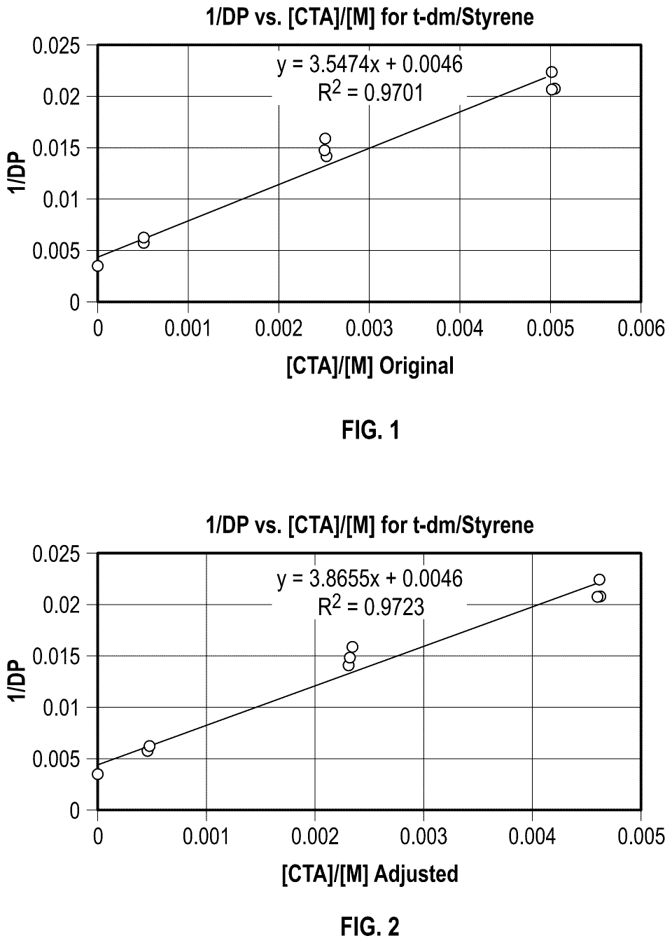

C_{S} is determined by creating polymerization experiments at various levels of CTA (including zero [CTA]), measuring the XN (DP) at those levels, and then plotting the data as 1/DP vs. [CTA]/[M] and obtaining C_{S} as the slope of a linear curve through the data. An example of such data analysis for TDM/styrene/AIBN at 70° C. developed in this example is depicted at FIG. 1. In FIG. 1, a value for C_{S} of 3.6 (unitless) was derived from the least squares fit of the data for the initial mercaptan and monomer concentrations.

The DP data resulting from the polymerization experiments represents the “cumulative” degree of polymerizations from the beginning of the reaction. It was necessary to measure the values of the mercaptan and the monomer concentrations over the same period of time that the polymer molecular weight was established. In this example, samples were withdrawn at reaction times when the monomer conversion levels were about 5% (see Tables 6-9, which include listings of all measured conversion levels at the times of sampling). The original (time=0) values of [CTA]/[M] were used to establish a Mayo plot for each mercaptan in order to obtain a first estimate of C_{S}. Then, the initial value of C_{S} was used to determine a more appropriate value of [CTA] at the mid-point of the conversion range over which the polymer molecular weights were created. The same was done for [M], and new, more appropriate, values of [CTA]/[M] were calculated for use in new Mayo plots, thus yielding more reliable values of the transfer constants.

Tables 6-9 depict the experimental conditions, the monomer conversions at which the samples were withdrawn for the analyses, ratios of mercaptan to monomer initial concentrations, and the molecular weight averages for M_{P} (peak), M_{N} (number), M_{W} (weight) and M_{Z} (Z) averages. In addition, the dispersity, Ð, is also displayed to indicate the breadth of the measured distributions.

TABLE 6
Experimental Details

| | Chain Transfer | CTA | Error in CTA | |
|---|---|---|---|---|
| Reaction No. | Agent (CTA) | (pph) | (pph) | [CTA]/[M] |
| 1 | TDM | 0.0982 | 0.00005 | 0.00050 |
| 2 | MMIO 1 | 0.0905 | 0.00005 | 0.00050 |
| 3 | MMIO 1 | 0.0911 | 0.00005 | 0.00050 |
| 4 | TNM | 0.0790 | 0.00005 | 0.00051 |
| 5 | TDM | 0.9894 | 0.00020 | 0.00505 |
| 6 | NDDM | 0.9825 | 0.00020 | 0.00507 |
| 7 | MMIO 4 | 0.1046 | 0.00005 | 0.00051 |
| 8 | MMIO 1 | 0.9123 | 0.00019 | 0.00505 |
| 9 | MMIO 2 | 0.0922 | 0.00005 | 0.00051 |
| 10 | MMIO 3 | 0.0922 | 0.00005 | 0.00051 |
| 11 | TDM | 0.4926 | 0.00011 | 0.00252 |
| 12 | MMIO 4 | 0.5236 | 0.00012 | 0.00253 |
| 13 | NDDM | 0.9540 | 0.00020 | 0.00492 |
| 14 | NDDM | 0.0981 | 0.00005 | 0.00051 |
| 15 | MMIO 3 | 0.4604 | 0.00010 | 0.00252 |
| 16 | MMIO 4 | 0.5227 | 0.00012 | 0.00253 |
| 17 | TMM | 0.8587 | 0.00018 | 0.00505 |
| 18 | TMM | 0.4265 | 0.00010 | 0.00251 |
| 19 | TNM | 0.7869 | 0.00017 | 0.00506 |
| 20 | TMM | 0.0857 | 0.00005 | 0.00050 |
| 21 | MMIO 1 | 0.9028 | 0.00019 | 0.00500 |
| 22 | NDDM | 0.0985 | 0.00005 | 0.00051 |
| 23 | TNM | 0.0778 | 0.00005 | 0.00050 |
| 24 | MMIO 3 | 0.9215 | 0.00019 | 0.00505 |
| 25 | TNM | 0.7842 | 0.00016 | 0.00504 |
| 26 | MMIO 2 | 0.4574 | 0.00010 | 0.00251 |
| 27 | MMIO 4 | 1.0475 | 0.00022 | 0.00506 |
| 28 | MMIO 2 | 0.9227 | 0.00019 | 0.00506 |
| 29 | TNM | 0.3923 | 0.00009 | 0.00252 |
| 30 | NDDM | 0.4891 | 0.00011 | 0.00252 |
| 31 | MMIO 1 | 0.4556 | 0.00010 | 0.00252 |

TABLE 7
GC and GPC Results

| Reaction | | | | | | | GPC Peak |
|---|---|---|---|---|---|---|---|
| No. | % Conversion | M_{P} | M_{N} | M_{W} | M_{Z} | Ð | Height |
| 1 | 6.11% | 53,141 | 17,448 | 59,283 | 112,493 | 3.4 | 127.3 |
| 2 | 6.42% | 27,514 | 10,959 | 31,418 | 60,645 | 2.9 | 98.2 |
| 3 | 5.93% | 27,920 | 9,500 | 30,427 | 60,411 | 3.2 | 117.9 |
| 4 | 6.26% | 61,511 | 14,768 | 58,940 | 116,602 | 4.0 | 124.7 |
| 5 | 5.44% | 9,810 | 4,929 | 11,041 | 20,243 | 2.2 | 97.4 |
| 6 | 5.72% | 3,348 | 2,607 | 3,973 | 5,923 | 1.5 | 62.9 |
| 7 | 6.00% | 21,614 | 8,279 | 23,477 | 45,872 | 2.8 | 119.4 |
| 8 | 5.10% | 3,498 | 2,996 | 4,742 | 7,233 | 1.6 | 64.6 |
| 9 | 5.26% | 44,586 | 15,178 | 46,272 | 86,484 | 3.0 | 108.2 |
| 10 | 5.69% | 37,408 | 11,898 | 38,719 | 76,294 | 3.3 | 116.2 |
| 11 | 5.43% | 18,402 | 7,201 | 19,886 | 38,678 | 2.8 | 112.7 |
| 12 | 5.58% | 4,897 | 3,679 | 6,683 | 11,127 | 1.8 | 78.0 |
| 13 | 4.38% | 2,788 | 2,880 | 4,295 | 6,284 | 1.5 | 52.8 |
| 14 | 4.57% | 23,597 | 9,948 | 28,591 | 56,119 | 2.9 | 100.9 |
| 15 | 5.05% | 9,810 | 5,616 | 12,166 | 21,719 | 2.2 | 91.0 |
| 16 | 5.01% | 5,042 | 3,738 | 6,639 | 10,950 | 1.8 | 71.7 |
| 17 | 4.79% | 3,111 | 2,814 | 4,058 | 5,686 | 1.4 | 56.0 |
| 18 | 4.72% | 21,145 | 7,932 | 21,123 | 39,977 | 2.7 | 110.5 |
| 19 | 5.16% | 9,955 | 5,607 | 12,475 | 22,463 | 2.2 | 94.2 |
| 20 | 5.53% | 66,179 | 18,664 | 64,707 | 125,936 | 3.5 | 109.2 |
| 21 | 3.36% | 4,019 | 2,947 | 4,573 | 6,820 | 1.6 | 48.3 |
| 22 | 5.45% | 26,917 | 9,103 | 27,317 | 54,645 | 3.0 | 105.3 |
| 23 | 5.38% | 58,441 | 17,233 | 62,119 | 121,490 | 3.6 | 111.1 |
| 24 | 4.92% | 5,230 | 3,888 | 6,789 | 10,938 | 1.7 | 69.3 |
| 25 | 0.54% | 12,040 | 4,758 | 12,672 | 25,846 | 2.7 | 21.4 |
| 26 | 5.07% | 12,766 | 6,396 | 14,830 | 26,818 | 2.3 | 87.9 |
| 27 | 4.48% | 3,157 | 2,662 | 3,803 | 5,363 | 1.4 | 41.5 |
| 28 | 4.93% | 6,234 | 4,598 | 8,824 | 15,052 | 1.9 | 87.9 |
| 29 | 5.37% | 19,368 | 8,782 | 22,616 | 42,272 | 2.6 | 94.0 |
| 30 | 7.56% | 7,008 | 4,618 | 8,404 | 13,895 | 1.8 | 75.4 |
| 31 | 6.66% | 7,935 | 4,921 | 9,747 | 16,915 | 2.0 | 85.0 |

TABLE 8
Additional Experimental Details

| | | | Experimental | |
|---|---|---|---|---|
| | | | Error in | |
| Reaction No. | CTA Name | CTA (pph) | CTA (pph) | [CTA]/[M] |
| 32 | TMM | 0.0852 | 0.00005 | 0.00050 |
| 33 | MMIO 4 | 0.0889 | 0.00005 | 0.00043 |
| 34 | TMM | 0.2553 | 0.00007 | 0.00150 |
| 35 | TMM | 0.4242 | 0.00010 | 0.00250 |
| 36 | TMM | 0.6766 | 0.00014 | 0.00398 |
| 37 | TMM | 0.8426 | 0.00017 | 0.00496 |
| 38 | MMIO 4 | 0.2663 | 0.00007 | 0.00129 |
| 39 | MMIO 4 | 0.4446 | 0.00010 | 0.00215 |
| 40 | MMIO 4 | 0.7058 | 0.00015 | 0.00341 |
| 41 | MMIO 4 | 0.8880 | 0.00018 | 0.00429 |

TABLE 9
GC and GPC Results

| | | | | | | | GPC |
|---|---|---|---|---|---|---|---|
| Reaction | | | | | | | Peak |
| No. | % Conversion | M_{P} | M_{N} | M_{W} | M_{Z} | Ð | Height |
| 32 | 6.01% | 55,120 | 14,668 | 52,870 | 100,108 | 3.6 | 135.2 |
| 33 | 6.64% | 21,532 | 9,617 | 25,049 | 46,480 | 2.6 | 128.6 |
| 34 | 6.21% | 29,450 | 10,066 | 28,277 | 52,383 | 2.8 | 135.2 |
| 35 | 6.20% | 17,818 | 7,461 | 19,017 | 35,255 | 2.5 | 117.3 |
| 36 | 6.75% | 12,025 | 5,858 | 13,711 | 25,580 | 2.3 | 119.2 |
| 37 | 5.39% | 8,728 | 5,119 | 11,180 | 20,374 | 2.2 | 99.8 |
| 38 | 5.95% | 8,602 | 5,097 | 10,904 | 19,546 | 2.1 | 103.3 |
| 39 | 5.03% | 5,055 | 3,848 | 7,261 | 12,517 | 1.9 | 91.6 |
| 40 | 5.04% | 3,564 | 2,893 | 4,650 | 7,226 | 1.6 | 70.0 |
| 41 | 5.31% | 137,896 | 60,452 | 135,635 | 223,929 | 2.2 | 118.1 |

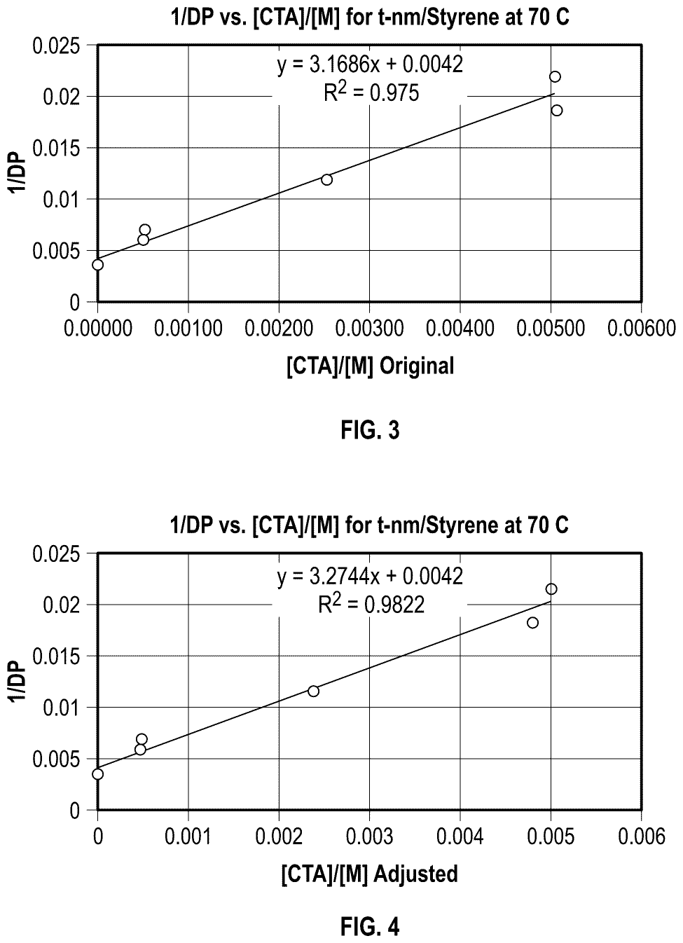

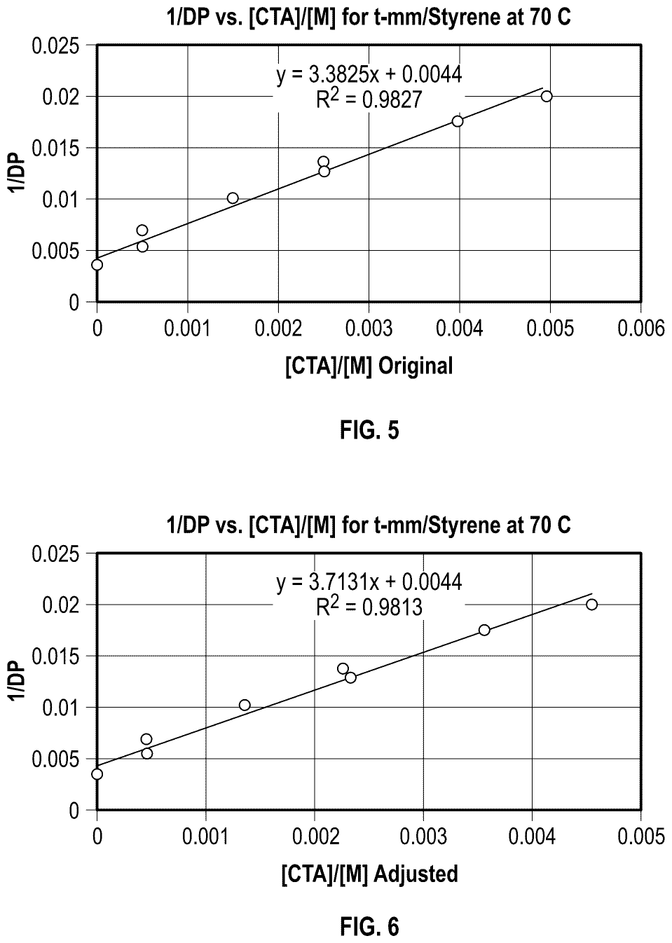

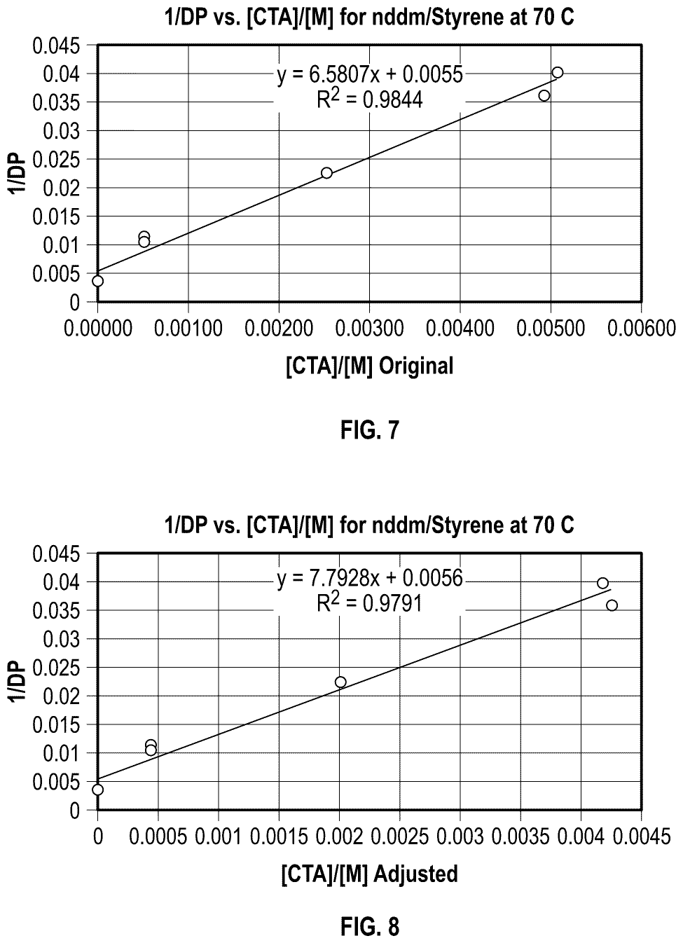

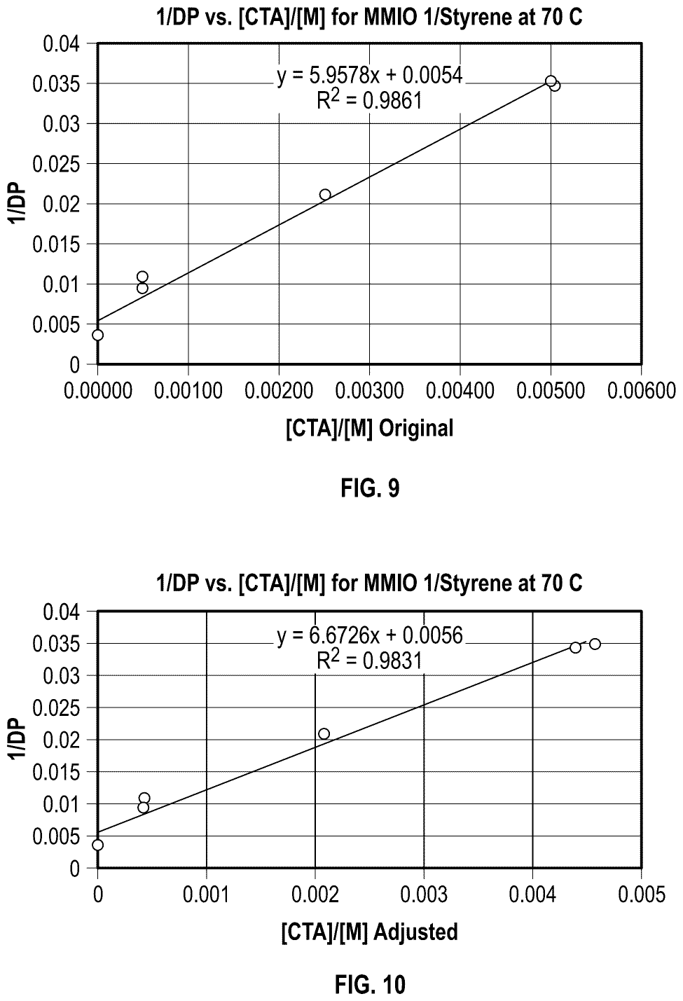

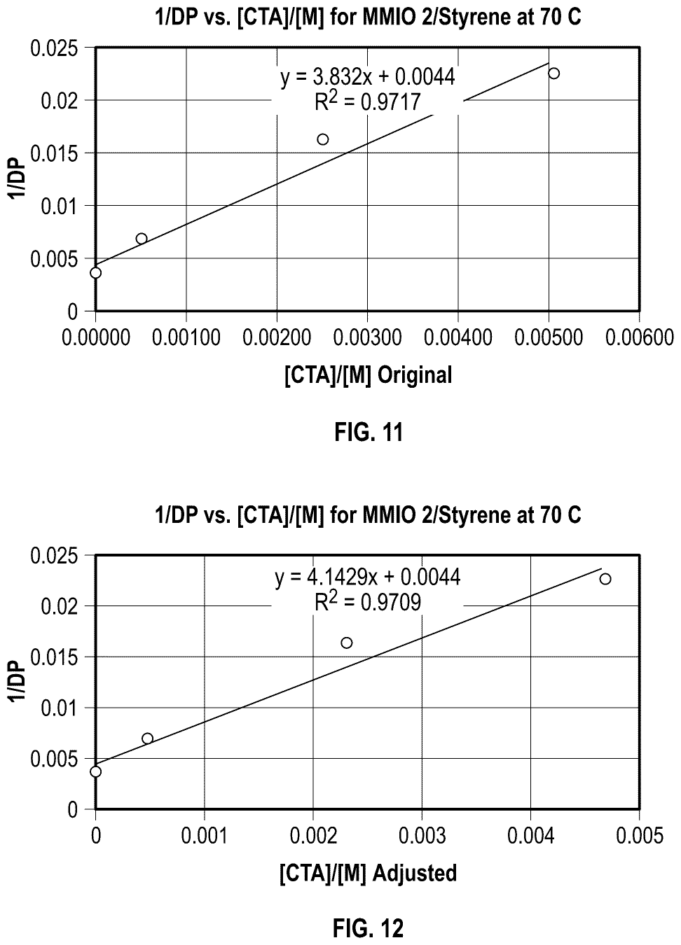

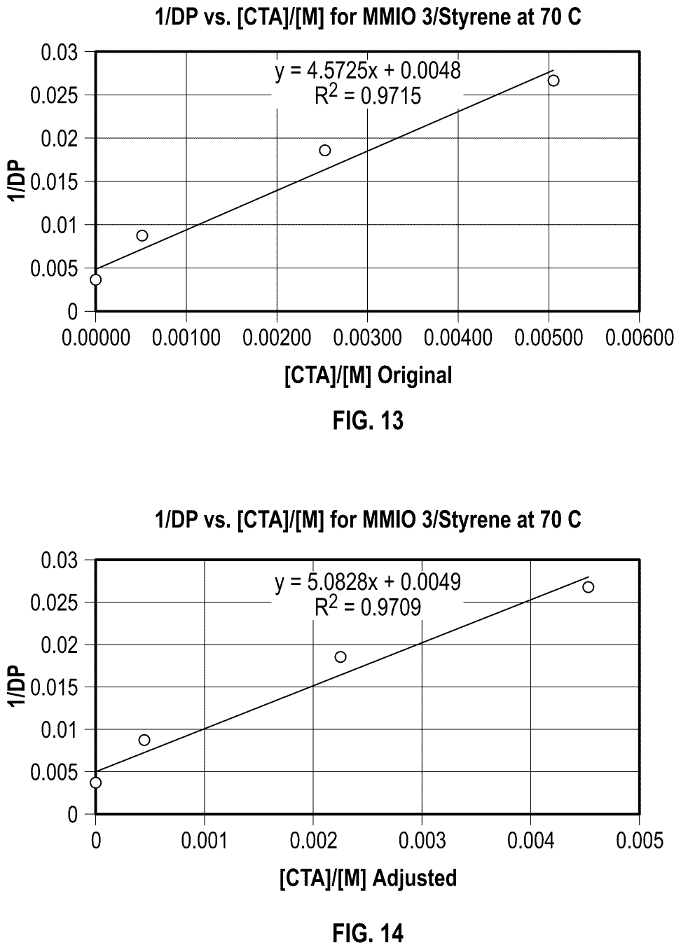

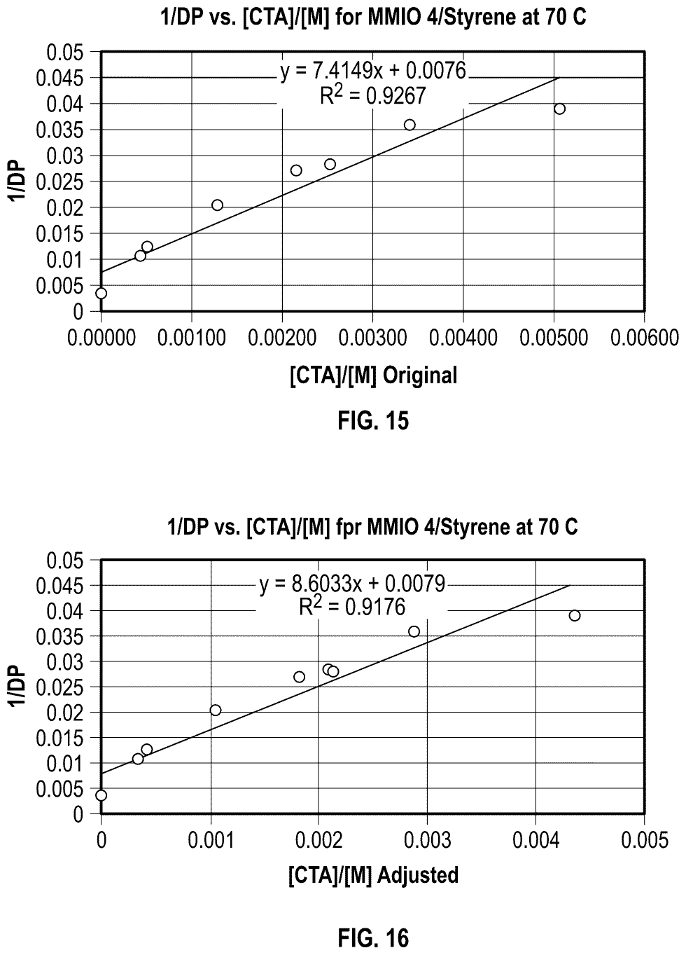

All 41 of the data sets of the foregoing tables were used to develop plots of 1/DP vs. [CTA]/[M], as described above and displayed, for example, in FIG. 1. These results are individually displayed at FIG. 1-FIG. 16.

As depicted at FIG. 1-FIG. 16, two Mayo plots were created for each mercaptan. The first employed [CTA]/[M] ratios using the initial (time=0) values for each concentration. From those plots, an initial estimate of the transfer constants was established. Those values were then used to determine the [CTA] level at a conversion mid-way between the start of the experiment and the time at which the sample was withdrawn. The same was done for the monomer concentrations, and then second Mayo plots were created. Both of these types of plots are shown in sequence for all of the mercaptans in the figures. The most appropriate value of the transfer constant, C_{S}, was determined from the linear slope of the second of each plot for each mercaptan. Included in each is the 95% confidence interval, CI.

In FIG. 2, 7 individual data points are plotted with sets of replicate experiments (some of the DP replicate data points are so close in value that they cannot be differentiated on the plot, which indicates strong experimental reproducibility). FIG. 2 also indicates the high quality of the linear fit to the data, and the determination of C_{S}=6.0 using the original mercaptan and monomer concentrations. Completing a 95% confidence interval analysis yields the following: C_{S}=6.0 +/−0.81.

The transfer constants determined for the eight mercaptan samples, with 95% confidence intervals, are reported in the following table.

TABLE 10
Transfer Constants

| Mercaptan | Transfer Constant to Styrene at 70° C. (Cs) |
|---|---|
| TDM | 3.9 +/− 0.5 |
| TNM | 3.3 +/− 0.5 |
| TMM | 3.7 +/− 0.5 |
| NDDM | 7.8 +/− 1.3 |
| MMIO 1 | 6.7 +/− 1.0 |
| MMIO 2 | 4.2 +/− 1.3 |
| MMIO 3 | 5.1 +/− 1.6 |
| MMIO 4 | 8.6 +/− 2.1 |

The data of this example provides a thorough study of the measured effects of a wide variety of mercaptan chain transfer agents on the molecular weight reductions for styrene polymerizations at 70° C. The extraction of the transfer constants, C_{S}, from the experiments for all of the mercaptans interacting with styrene polymer radicals used the Mayo equation approach, and the resulting values were clearly within the range of expectations.

All mercaptans of this example led to substantial decreases of polymer average molecular weights.

The transfer constants developed in this work are specific to styrene monomer and its polymerization at 70° C. However, easily discernible trends in the data demonstrate that these same values of C_{S} can safely be extended to other monomers, such as those with free radicals having a reactivity similar to that of the polystyrene free radical. For example, the same values of C_{S} provided in this example could be safely extended to polybutadiene.

Regarding the temperature effect on C_{S}, it is known that there can be a slight increase in the magnitude of transfer constants with increasing reaction temperature, so the same behavior would be expected for the values determined from this example.

### Example 2—Emulsion Polymerizations

In this example, a series of emulsion polymerization reactions were conducted and evaluated. As in Example 1, styrene was used as the monomer, and the mercaptan choices were TDM, MMIO 2 and MMIO 4. The basic emulsion polymerization composition used is depicted in the following table:

TABLE 11
Emulsion Polymerization Parameters

| Component | Level (parts) |
|---|---|
| Water | 180 |
| Surfactant (sodium dodecyl sulfate, SDS) | 3.0 |
| Initiator (sodium persulfate, NaPS) | 0.2 |
| Styrene monomer | 100 |
| Mercaptan (various ones) | 0.25, 0.5% on monomer |

The reactions were conducted in lab scale vessels and controlled at 70° C. Samples of the reacting latex were withdrawn periodically, quenched to halt the reaction, and subsequently analyzed for the degree of monomer conversion and for the polymer molecular characteristics.

The experiments permitted collection of M_{W} vs. monomer conversion EP data. The effects of CTA transport from the emulsified droplets was determined through the water and into the polymer particles, as well as in the chemical reactivity of the mercaptans (the transfer constants, C_{S}).

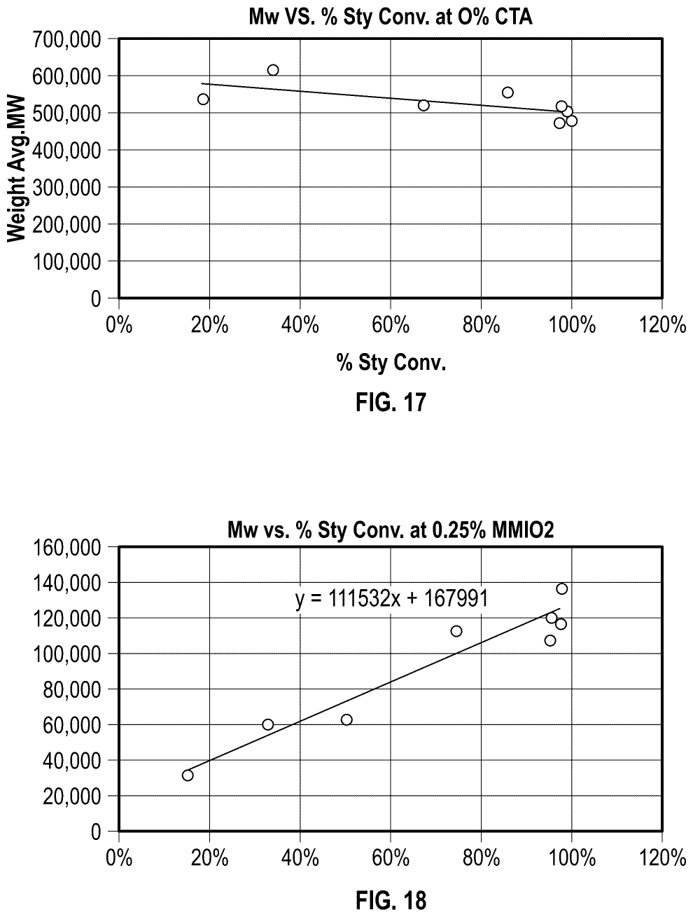

To gain perspective of the M_{W} reducing power of any of the chain transfer agents, an EP experiment was conducted with no chain transfer agent, and the results are depicted in FIG. 17. FIG. 17 shows a very high M_{W} at all conversions, which is typical of EP with no added M_{W} control. The slight decrease in M_{W} with conversion, especially after 35% conversion, is likely due to the decreasing monomer concentration in the latex particles after all of the emulsified monomer droplets disappeared, which is inherent in the mechanism of EP. A role of chain transfer agents is to reduce the magnitude of these high weight average molecular weights. In contrast, FIG. 20 depicts the M_{W} vs. conversion results obtained when TDM was used at 0.25% on monomer. The M_{W} values decreased by a factor of about 5 and were rather constant with conversion.

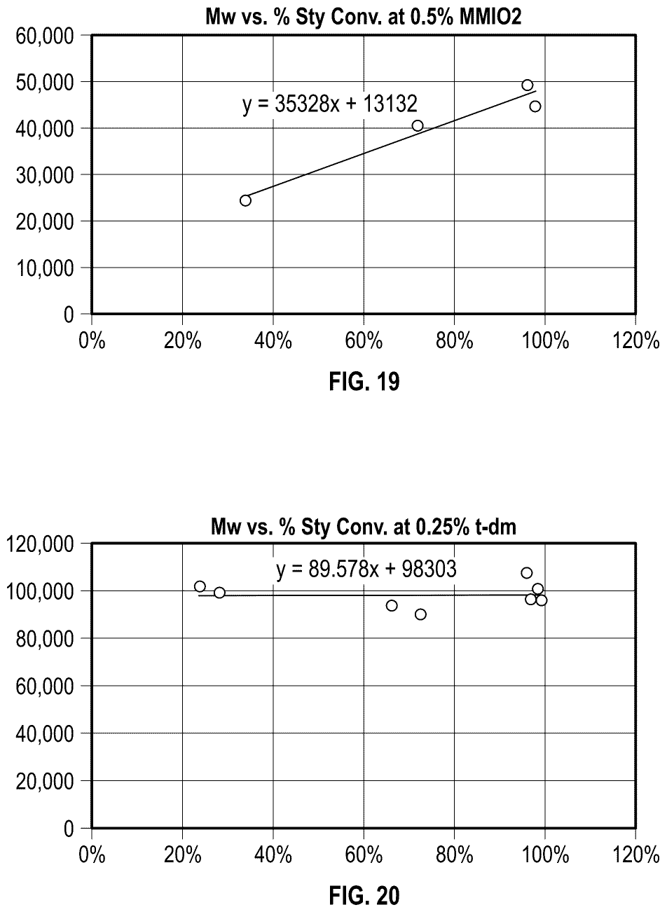

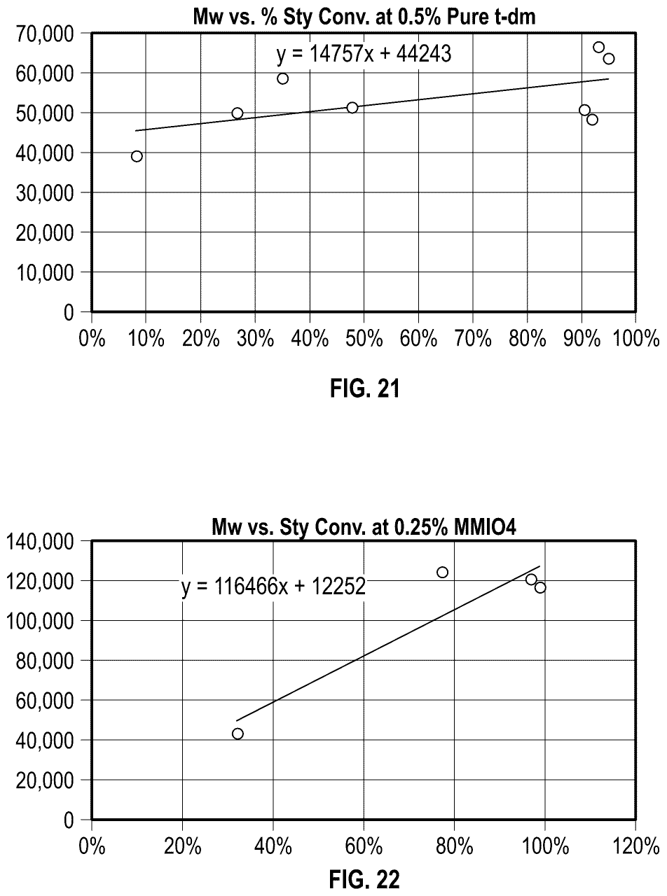

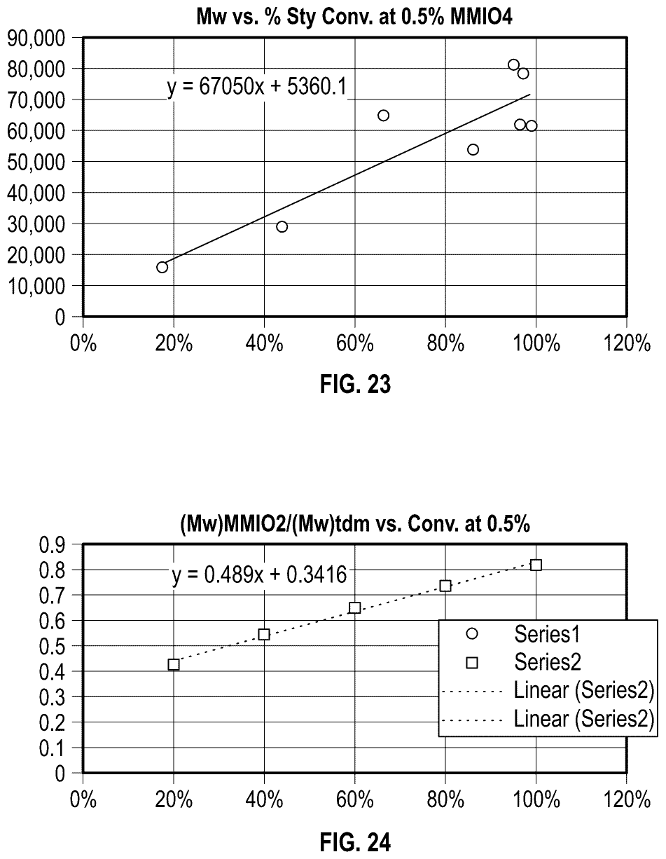

The EP results for MMIO 2 and MMIO 4 mercaptans, also at 0.25%, are depicted in FIG. 18 and FIG. 22. It was apparent that the trend of these data with conversion was markedly different than that of TDM, although M_{W} at the end of the polymerization reactions was not very different. The strong variation with monomer conversion in the M_{W} data for the MMIO series mercaptans at the 0.25% levels is an indication that these mercaptans are significantly more water soluble than TDM and were likely transported more quickly to the latex particles than the TDM. This likely led to strong M_{W} reduction early in the reaction, which subsided later in the reaction. This appears to be due to a high mercaptan consumption rate at low conversions, resulting in a lack of supply in the latter stages of the reaction. The corresponding data for the three mercaptans at the 0.5% level are shown at FIG. 19, FIG. 21, and FIG. 23.

In view of the results for the MMIO series mercaptans and their conversion dependent profiles, the advantages of mixed mercaptan systems, particularly MMIO 2 and TDM, were realized and tested. Both of these agents have very similar transfer constants (C_{S}˜4), but likely quite different water solubilities. Due to the fact that the MMIO 2 can be the predominate actor during the early part of the EP reaction, and the TDM can be the primary actor later in the polymerization, a series of experiments was carried out with a total mercaptan loading of 0.5 wt. %, but with different ratios of these two mercaptans.

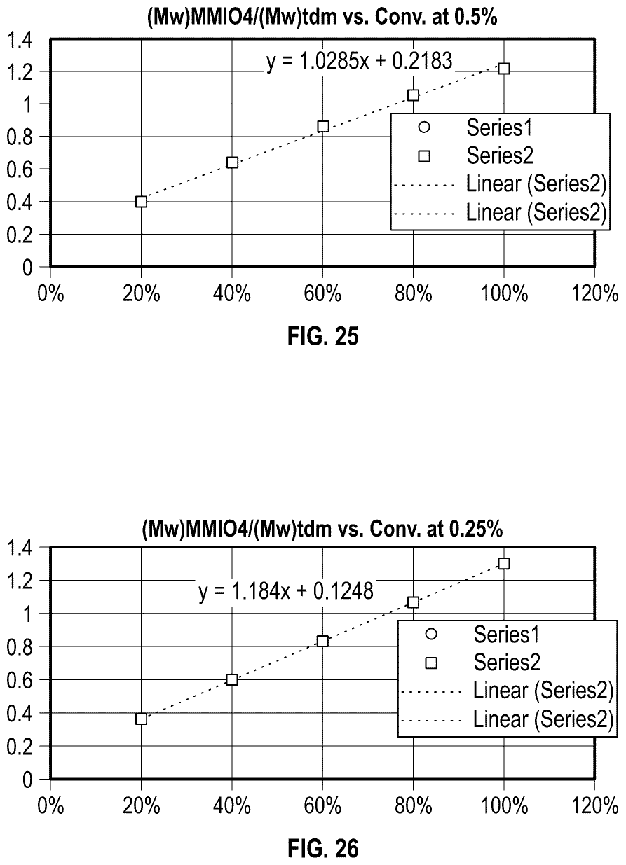

FIG. 24-FIG. 26 show the M_{W} versus conversion data expressed as the M_{W} of a particular mercaptan divided by that for TDM.

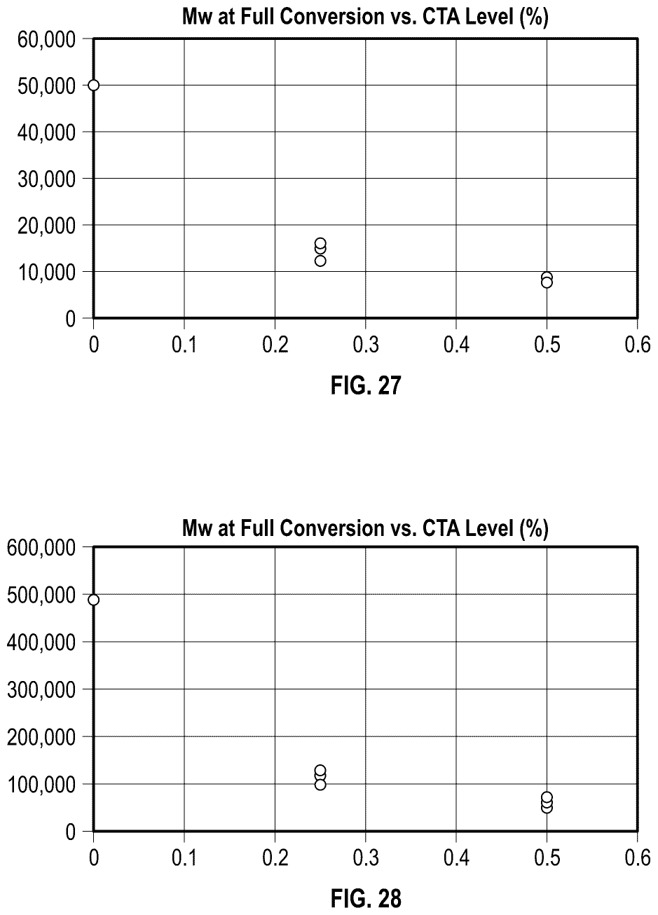

FIG. 27 and FIG. 28 depict the average molecular weights at full monomer conversion (i.e., at the end of the reaction) for all of the single mercaptan emulsion polymerization experiments. FIG. 27 plots the number average molecular weight, and FIG. 28 depicts the weight average molecular weight.

Experiments were carried out at MMIO 2:TDM ratios of 3:1, 1:1, and 1:3 (75, 50 and 25% MMIO 2 in the mixture). The total mercaptan load was kept at 0.5% on monomer for consistency.

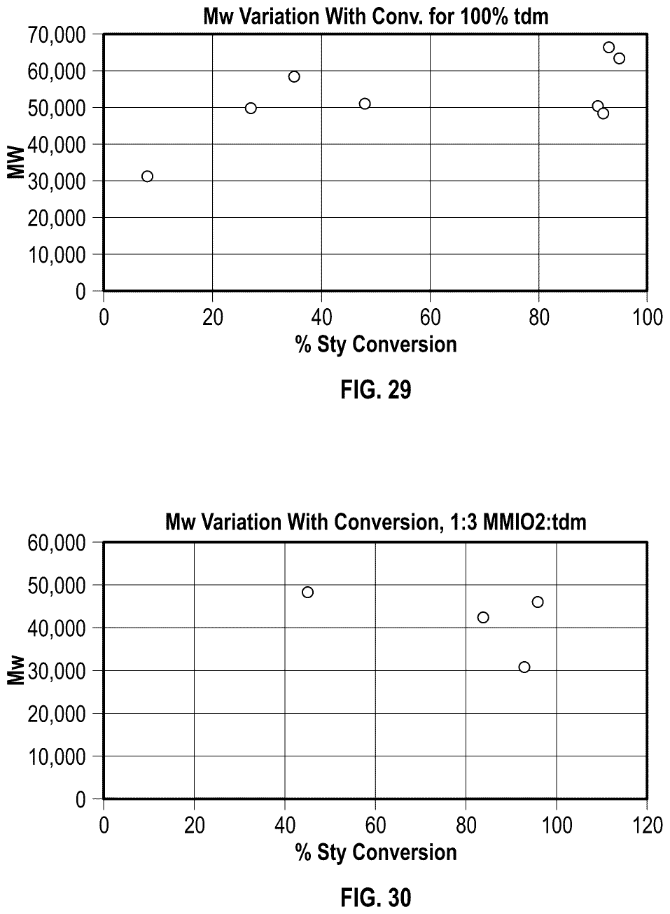

FIG. 32 depicts the weight average (M_{W}) versus conversion profiles for a 3:1 (75 percent) ratio of MMIO 2:TDM, with a total loading of 0.5% on monomer. Of note in this figure is (a) the M_{W} variation with conversion was not strong, and (b) the final M_{W} was significantly less than that resulting from the use of pure TDM at a 0.5% loading on monomer (see FIG. 29).

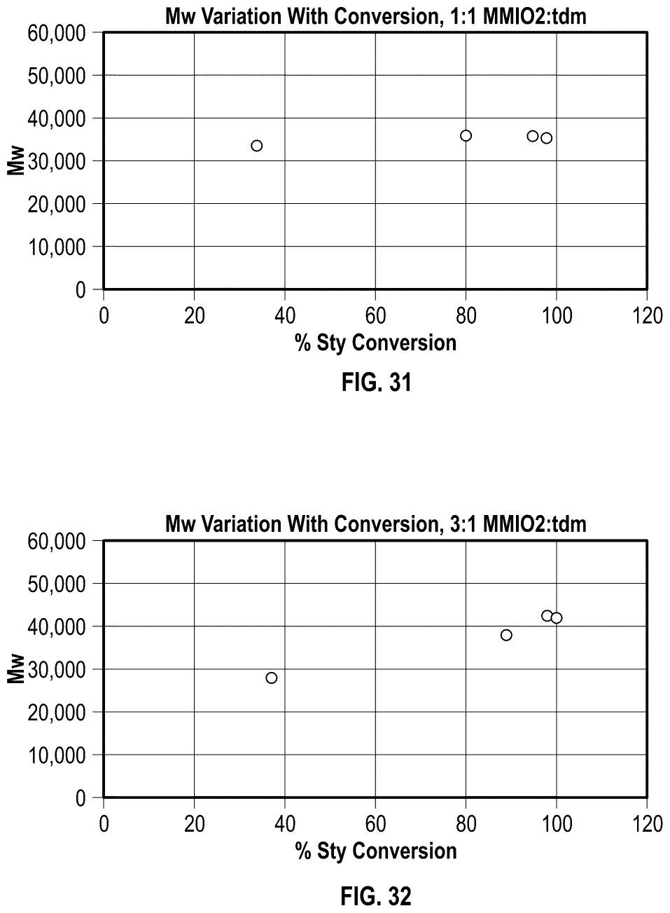

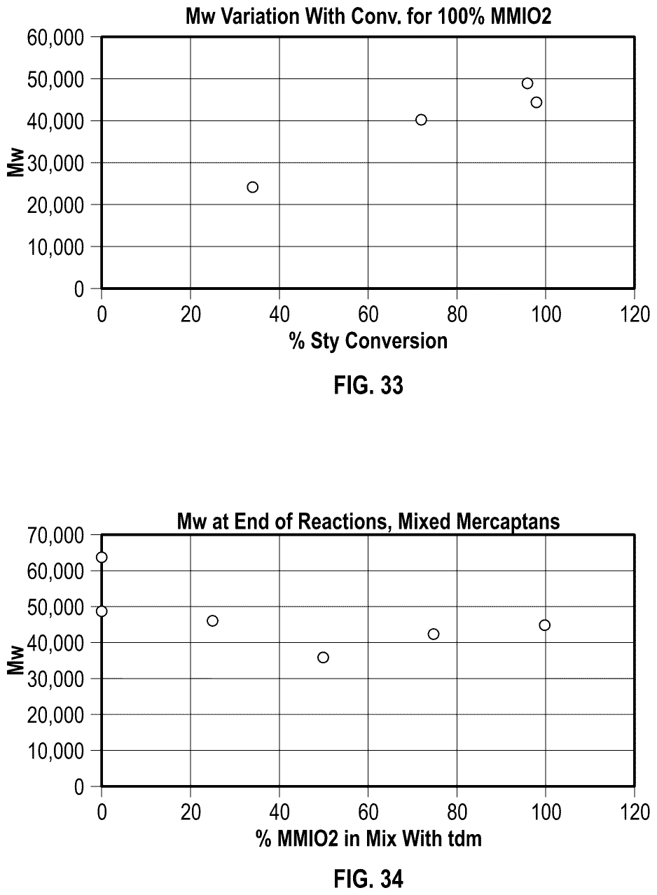

FIG. 31 shows the results for the 1:1 ratio of the mercaptans. For this mixture, the conversion dependency of the M_{W} was suppressed, and the final M_{W} was substantially lower than that obtained using pure TDM or MMIO 2.

Other mixed mercaptan experiments were conducted with a ratio of MMIO 2:TDM at 1:3, or 25% of MMIO 2 in the mercaptan mix. These results are depicted in FIG. 30. The conversion dependency was restricted, and the final M_{W} was somewhat lower than that of pure TDM.

FIG. 34 depicts the final (full conversion) M_{W} results for all of the mixed mercaptan experiments. The two data points at 0% MMIO 2 were the results of two repeat experiments using pure TDM These data indicate that MMIO 2 can be mixed with TDM in a variety of proportions, each resulting in about the same final weight average M_{W}.

In these examples, all of the MMIO series mercaptans led to substantial decreases of polymer average molecular weights. The transfer constants (C_{S}) for these mercaptans at 70° C. varied from about 4 to about 9 (for comparison, the C_{S} for TDM is 4).

While the transfer constants refer to the actual chemical reactivity of the thiols towards polymeric free radicals, their usefulness in polymerization reactions can depend upon the particular reaction system of interest, with emulsion polymerization being one of the most important. From the EP experiments of these examples, the data indicate that the MMIO 2 and MMIO 4 mercaptans likely have significantly greater water solubility than TDM, particularly MMIO 4. This likely resulted in the M_{W} being more dependent on the styrene monomer conversion during the EP experiments with those mercaptans. However, experiments conducted with mixtures of MMIO 2 with TDM (at a total mercaptan loading of 0.5% on monomer) yielded M_{W} versus conversion results that dramatically lessened that conversion dependency. Further, nearly all mixture ratios of MMIO 2 and TDM resulted in very close to the same final, full conversion weight average molecular weights.

### ASPECTS

The following is a non-limiting list of Aspects:

Aspect 1. A composition, such as a chain transfer agent composition, comprising (consisting essentially of, or consisting of):
(i) a first component comprising (consisting essentially of, or consisting of) tert-dodecyl mercaptan, tert-nonyl mercaptan, n-dodecyl mercaptan, or a combination thereof, such as a mixture of tert-dodecyl mercaptan and tert-nonyl mercaptan (for example, the mixture of tert-dodecyl mercaptan and tert-nonyl mercaptan can comprise, consist essentially of, or consist of tert-dodecyl mercaptan and tert-nonyl mercaptan, and when the mixture “consists essentially of” tert-dodecyl mercaptan and tert-nonyl mercaptan, the mixture can include other compounds that do not include a mercaptan functional group); and
(ii) a second component comprising (consisting essentially of, or consisting of) one or more alkyl mercaptans, one or more alkyl sulfides, or a combination thereof, such as, respectively, one or more branched alkyl mercaptans, one or more branched alkyl sulfides, or a combination thereof; wherein, optionally, at least one of the one or more branched alkyl mercaptans of the second component is not tert-dodecyl mercaptan, is not tert-nonyl mercaptan, or is not tert-dodecyl mercaptan and is not tert-nonyl mercaptan.

Aspect 2. The composition of Aspect 1, wherein the first component and the second component are present at a weight ratio (first component: second component) of about 10:90 to about 99:1, about 20:80 to about 99:1, about 30:70 to about 99:1, about 40:60 to about 99:1, about 50:50 to about 99:1, about 60:40 to about 99:1, about 70:30 to about 99:1, about 80:20 to about 99:1, or about 90:10 to about 99:1; or
wherein the second component and the first component are present at a weight ratio (second component: first component) of about 10:90 to about 99:1, about 20:80 to about 99:1, about 30:70 to about 99:1, about 40:60 to about 99:1, about 50:50 to about 99:1, about 60:40 to about 99:1, about 70:30 to about 99:1, about 80:20 to about 99:1, or about 90:10 to about 99:1.

Aspect 3. The chain transfer agent of Aspect 1 or 2, wherein the one or more alkyl mercaptans comprises (consists essentially of, or consists of) one or more C₅ to C₂₀ mercaptans, one or more C₅ to C₁₅ mercaptans, one or more C₈ to C₁₂ mercaptans, one or more C₉ to C₁₁ mercaptans, or one or more C₁₀ mercaptans, such as, respectively, one or more branched C₅ to C₂₀ mercaptans, one or more branched C₅ to C₁₅ mercaptans, one or more branched C₈ to C₁₂ mercaptans, one or more branched C₉ to C₁₁ mercaptans, or one or more branched C₁₀ mercaptans.

Aspect 4. The chain transfer agent of any one of the preceding Aspects, wherein the one or more alkyl sulfides comprises (consists essentially of, or consists of) one or more C₁₀ to C₄₀ sulfides, one or more C₁₀ to C₃₀ sulfides, or one or more C₂₀ sulfides, such as, respectively, one or more branched C₁₀ to C₄₀ sulfides, one or more branched C₁₀ to C₃₀ sulfides, or one or more branched C₂₀ sulfides.

Aspect 5. The composition of any of the preceding Aspects, wherein the one or more branched alkyl mercaptans, the one or more branched alkyl sulfides, or a combination thereof are the reaction products of contacting hydrogen sulfide (H₂S), a feedstock comprising one or more branched C₅ to C₂₀ monoolefins, and an initiating agent and/or a catalyst.

Aspect 6. The composition of Aspect 5, wherein the one or more branched C₅ to C₂₀ monoolefins comprise (consist essentially of or consist of) branched C₁₀ monoolefins.

Aspect 7. The composition of Aspect 6, wherein the branched C₁₀ monoolefins comprise (consist essentially of or consist of) 5-methyl-1-nonene, 3-propyl-1-heptene, 4-ethyl-1-octene, 2-butyl-1-hexene, or combinations thereof; and wherein the crude composition comprises (consists essentially of or consists of) branched C₁₀ mercaptans and branched C₂₀ sulfides.

Aspect 8. The composition of any one of the preceding Aspects,
(i) wherein the one or more alkyl mercaptans comprise (consist essentially of, or consist of) one or more mercaptans of the following formula—

R—SH;
wherein each R is independently selected from a C₅ to C₂₀ alkyl, a C₅ to C₁₅ alkyl, a C₈ to C₁₂ alkyl, a C₉ to C₁₁ alkyl, or a C₁₀ alkyl, such as, respectively, a branched C₅ to C₂₀ alkyl, a branched C₅ to C₁₅ alkyl, a branched C₈ to C₁₂ alkyl, a branched C₉ to C₁₁ alkyl, or a branched C₁₀ alkyl; or
(ii) wherein the one or more branched alkyl mercaptans comprise (consist essentially of, or consist of) a compound Structure A, a compound of Structure B, a compound of Structure C, a compound of Structure D, a compound of Structure E, a compound of Structure F, a compound of Structure G, a compound of Structure H, or a combination thereof:

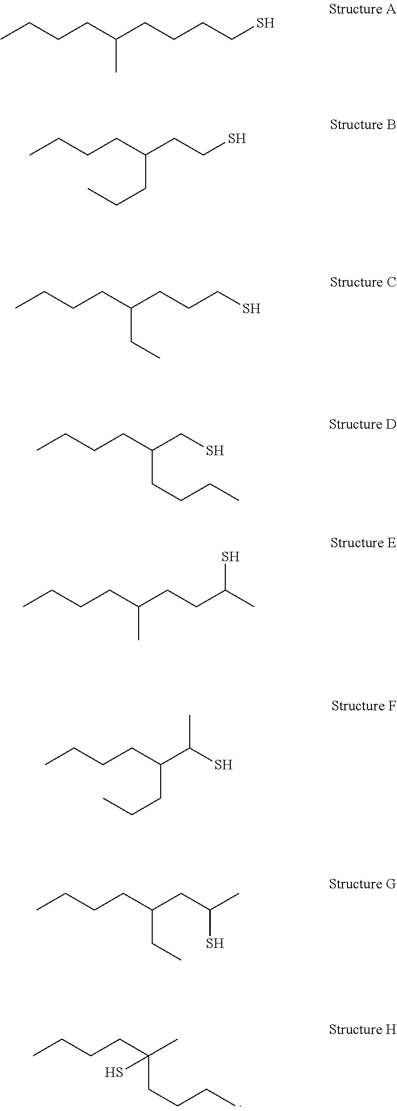

Aspect 9. The composition of any one of the preceding Aspects, wherein the one or more alkyl sulfides comprise (consist essentially of, or consist of) one or more compounds of the following formula—

R¹—S—R²;
(i) wherein each R¹ and R² is independently selected from a C₅ to C₂₀ alkyl, a C₅ to C₁₅ alkyl, a C₈ to C₁₂ alkyl, a C₉ to C₁₁ alkyl, or a C₁₀ alkyl, such as, respectively, a branched C₅ to C₂₀ alkyl, a branched C₅ to C₁₅ alkyl, a branched C₈ to C₁₂ alkyl, a branched C₉ to C₁₁ alkyl, or a branched C₁₀ alkyl; or
(ii) wherein R¹ and R² are independently selected from the group consisting of the following:

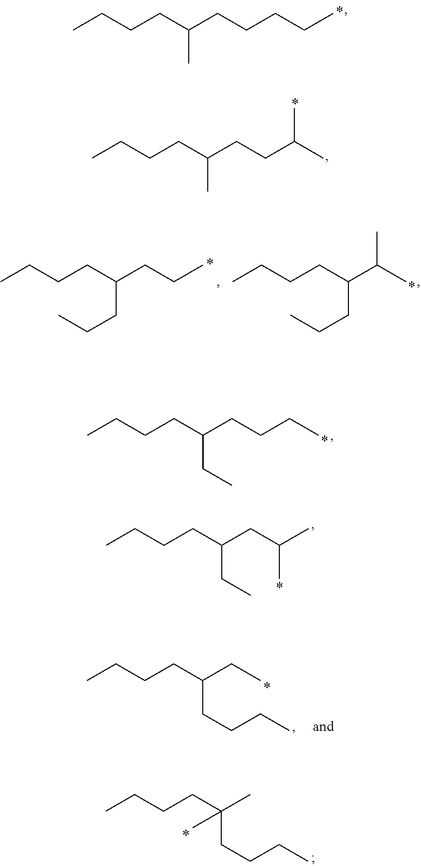

wherein “*” designates attachment points of the “S” atom.

Aspect 10. The composition of any one of the preceding Aspects, wherein the second component comprises (consists essentially of, or consists of) (i) one or more C₁₀ mercaptans, such as one or more branched C₁₀ mercaptans and (ii) one or more C₁₂ mercaptans, one or more C₁₄ mercaptans, one or more C₁₆ mercaptans, one or more C₁₈ mercaptans, or a combination thereof, such as, respectively, one or more branched C₁₂ mercaptans, one or more branched C₁₄ mercaptans, one or more branched C₁₆ mercaptans, one or more branched C₁₈ mercaptans, or a combination thereof.

Aspect 11. The composition of any one of the preceding Aspects, wherein the one or more alkyl mercaptans, such as the one or more branched alkyl mercaptans, are present at an amount of at least about 50 wt %, at least about 60 wt %, at least about 70 wt %, at least about 80 wt %, at least about 85 wt %, or at least about 90 wt %, based on the weight of the second component.

Aspect 12. The composition of any one of the preceding Aspects, wherein the one or more alkyl sulfides, such as the one or more branched alkyl sulfides, are present at an amount of less than about 40 wt %, less than about 30 wt %, less than about 20 wt %, less than about 15 wt %, or less than about 10 wt %, based on the weight of the second component.

Aspect 13. The composition of any one of the preceding Aspects, wherein the second component comprises (consists essentially of, or consists of) MMIO 1.

Aspect 14. The composition of any one of the preceding Aspects, wherein the second component comprises (consists essentially of, or consists of) MMIO 2.

Aspect 15. The composition of any one of the preceding Aspects, wherein the second component comprises (consists essentially of, or consists of) MMIO 3.

Aspect 16. The composition of any one of the preceding Aspects, wherein the second component comprises (consists essentially of, or consists of) MMIO 4.

Aspect 17. The composition of any one of the preceding Aspects, wherein the first component comprises (consists essentially of, or consists of) tert-dodecyl mercaptan, and the second component comprises (consists essentially of, or consists of) MMIO 1, MMIO 2, MMIO 3, MMIO 4, or a combination thereof.

Aspect 18. The composition of any one of the preceding Aspects, wherein the first component comprises (consists essentially of, or consists of) tert-nonyl mercaptan, and the second component comprises (consists essentially of, or consists of) MMIO 1, MMIO 2, MMIO 3, MMIO 4, or a combination thereof.

Aspect 19. The composition of any one of the preceding Aspects, wherein the first component comprises (consists essentially of, or consists of) n-dodecyl mercaptan, and the second component comprises (consists essentially of, or consists of) MMIO 1, MMIO 2, MMIO 3, MMIO 4, or a combination thereof.

Aspect 20. The composition of any one of the preceding Aspects, wherein the first component comprises (consists essentially of, or consists of) a mixture of tert-dodecyl mercaptan and tert-nonyl mercaptan, and the second component comprises (consists essentially of, or consists of) MMIO 1, MMIO 2, MMIO 3, MMIO 4, or a combination thereof.

Aspect 21. A polymerization reaction mixture, such as an emulsion polymerization reaction mixture, comprising (consisting essentially of, or consisting of) the chain transfer agent composition of any one of Aspects 1 to 20; optionally one or more monomers; optionally one or more surfactants; optionally one or more polymerization initiators; and optionally water.

Aspect 22. The polymerization reaction mixture of Aspect 21, wherein the chain transfer agent composition is present in an amount of about 0.01 wt % to about 10 wt %, about 0.01 wt. % to about 5 wt. %, about 0.01 wt. % to about 4 wt. %, about 0.01 wt. % to about 3 wt. %, about 0.01 wt. % to about 2 wt. %, about 0.01 wt. % to about 1 wt. %, based on a total weight of the emulsion polymerization mixture.

Aspect 23. The polymerization reaction mixture of Aspect 21 or 22, wherein the second component is present in an amount of at least 10 wt. %, at least 20 wt. %, or at least 30 wt. % based on a total weight of the chain transfer agent composition.

Aspect 24. The polymerization reaction mixture of any of the preceding Aspects, wherein the emulsion polymerization mixture comprises one or more monomer, one or more surfactant, one or more polymerization initiator, and water.

Aspect 25. The polymerization reaction mixture of any of the preceding Aspects, wherein the one or more monomers is capable of free radical polymerization.

Aspect 26. A method comprising providing the emulsion polymerization mixture of any of the preceding Aspects; and optionally isolating a polymer product from the emulsion polymerization mixture.

1. A chain transfer agent composition comprising:
a first component comprising tert-dodecyl mercaptan, tert-nonyl mercaptan, n-dodecyl mercaptan, or a mixture of tert-dodecyl mercaptan and tert-nonyl mercaptan; and
a second component comprising one or more branched alkyl mercaptans, one or more branched alkyl sulfides, or a combination thereof;
wherein at least one of the one or more branched alkyl mercaptans of the second component is not tert-dodecyl mercaptan and is not tert-nonyl mercaptan.

2. The chain transfer agent composition of claim 1, wherein the first component comprises tert-dodecyl mercaptan, tert-nonyl mercaptan, n-dodecyl mercaptan, n-dodecyl mercaptan, or a combination thereof.

3. The chain transfer agent composition of claim 1, wherein the first component comprises n-dodecyl mercaptan.

4. The chain transfer agent composition of claim 1, wherein the one or more branched alkyl mercaptans comprises one or more branched C₅ to C₈ mercaptans, one or more branched C₁₀ to C₁₁ mercaptans, one or more branched C₁₃ to C₂₀ mercaptans, or a combination thereof.

5. The chain transfer agent composition of claim 1, wherein the one or more branched alkyl mercaptans, the one or more branched alkyl sulfides, or a combination thereof are the reaction products of contacting hydrogen sulfide (H₂S), a feedstock comprising one or more branched C₅ to C₂₀ monoolefins, and an initiating agent or a catalyst.

6. The chain transfer agent composition of claim 5, wherein the one or more branched C₅ to C₂₀ monoolefins comprise branched C₁₀ monoolefins.

7. The chain transfer agent composition of claim 6, wherein the branched C₁₀ monoolefins comprise 5-methyl-1-nonene, 3-propyl-1-heptene, 4-ethyl-1-octene, 2-butyl-1-hexene, or a combination thereof.

8. The chain transfer agent composition of claim 1, wherein the one or more branched alkyl mercaptans comprise a compound of Structure A, a compound of Structure B, a compound of Structure C, a compound of Structure D, a compound of Structure E, a compound of Structure F, a compound of Structure G, a compound of Structure H, or a combination thereof:

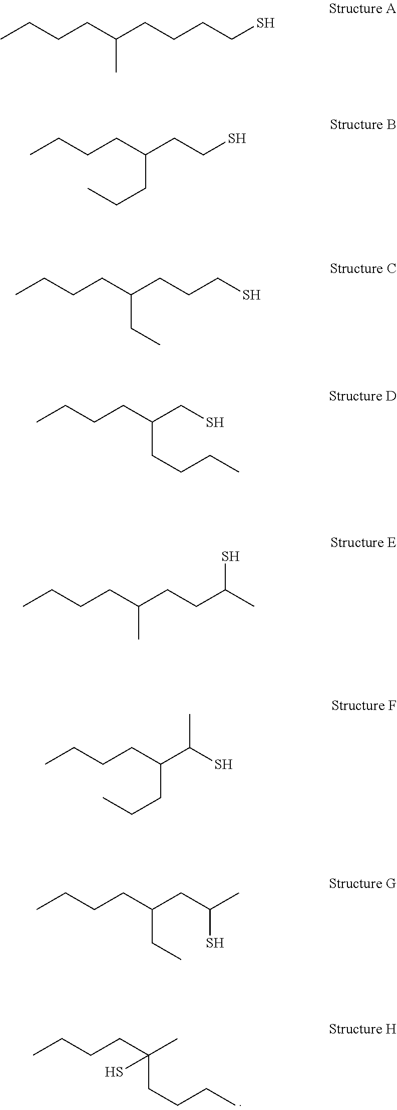

9. The chain transfer agent composition of claim 1, wherein the first component and the second component are present at a weight ratio (first component: second component) of about 10:90 to about 99:1.

10. The chain transfer agent composition of claim 1, wherein the first component and the second component are present at a weight ratio (first component:second component) of about 50:50 to about 99:1.

11. The chain transfer agent composition of claim 1, wherein the first component and the second component are present at a weight ratio (first component:second component) of about 80:20 to about 99:1.

12. The chain transfer agent composition of claim 1, wherein the one or more branched alkyl sulfides comprise one or more branched C₁₀ to C₄₀ sulfides.

13. The chain transfer agent composition of claim 1, wherein the one or more branched alkyl sulfides comprise one or more branched C₂₀ sulfides.

14. The chain transfer agent composition of claim 1, wherein the one or more branched alkyl sulfides comprise one or more compounds of the formula—
R¹—S—R²;
wherein R¹ and R² are independently selected from the group consisting of

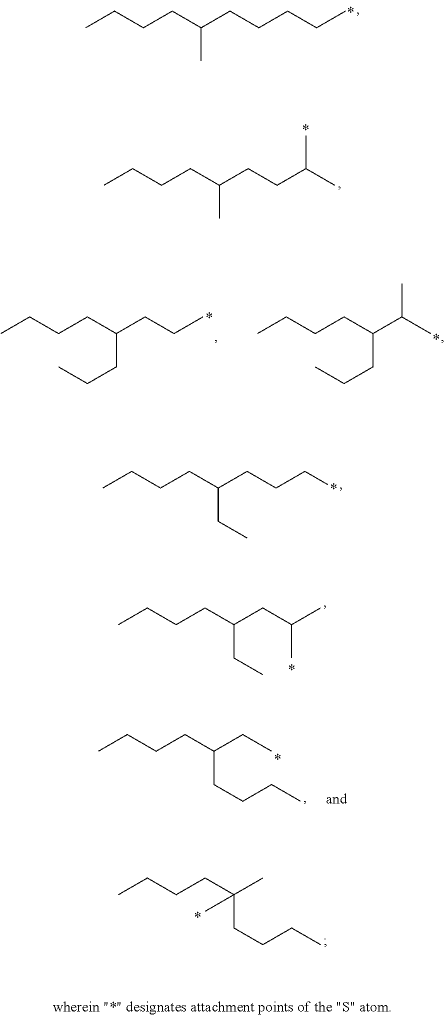

15. The chain transfer agent composition of claim 1, wherein the second component comprises (i) one or more branched C₁₀ mercaptans, and (ii) one or more C₁₂ mercaptans, one or more C₁₄ mercaptans, one or more C₁₆ mercaptans, one or more C₁₈ mercaptans, or a combination thereof.

16. The chain transfer agent composition of claim 1, wherein the one or more branched alkyl mercaptans are present at an amount of at least about 60 wt. %, based on the weight of the second component.

17. The chain transfer agent composition of claim 1, wherein the one or more branched alkyl mercaptans are present at an amount of at least about 90 wt. %, based on the weight of the second component.

18. The chain transfer agent composition of claim 1, wherein the one or more branched alkyl sulfides are present at an amount of less than about 40 wt. %, based on the weight of the second component.

19. An emulsion polymerization reaction mixture comprising:
the chain transfer agent composition of any one of claim 1;
one or more monomers;
one or more polymerization initiators;
optionally one or more surfactants; and
water;
wherein the chain transfer agent composition is present at an amount of about 0.01 wt % to about 5 wt %, based on a total weight of the emulsion polymerization mixture.

20. The emulsion polymerization reaction mixture of claim 19, wherein the second component is present at an amount of at least 10 wt %, based on a total weight of the chain transfer agent composition.

21. The emulsion polymerization reaction mixture of claim 19, wherein the second component is present at an amount of at least 30 wt %, based on a total weight of the chain transfer agent composition.

22. A method of polymerization, the method comprising:
providing the emulsion polymerization reaction mixture of claim 19; and
isolating a polymer product from the emulsion polymerization reaction mixture.
